(19) United States
(12) Patent Application Publication
(10) Pub. No.: US 2008/0131907 A1
(43) Pub. Date: Jun. 5, 2008

(54) ASSAYS FOR DETECTING NATIVE-STATE PROTEINS AND IDENTIFYING COMPOUNDS THAT MODULATE THE STABILITY OF NATIVE-STATE PROTEINS

(75) Inventors: Lan Wang, Newton, MA (US); Christine Bulawa, Arlington, MA (US)

Correspondence Address: FISH & RICHARDSON PC, P.O. BOX 1022, MINNEAPOLIS, MN, 55440-1022, US

(73) Assignee: FoldRx Pharmaceuticals, Inc., Cambridge, MA (US)

(21) Appl. No.: 11/855,863

(22) Filed: Sep. 14, 2007

Related U.S. Application Data
(60) Provisional application No. 60/825,809, filed on Sep. 15, 2006.

(51) Int. Cl. G01N 33/573; G01N 33/566; G01N 33/53; G01N 33/543

(52) U.S. Cl. 435/7.1; 436/518; 436/501; 435/7.4

(57) ABSTRACT

This invention relates to assays for detecting native-state proteins and to screening methods to identify compounds that modulate the stability of the native-state of proteins. Compounds identified by such screens can be used to treat diseases associated with protein misfolding, such as Alzheimer's Disease, familial amyloidotic polyneuropathies, and lysosomal storage diseases.

### CROSS-REFERENCE TO RELATED APPLICATIONS

This application claims priority from U.S. Provisional Application No. 60/825,809, filed Sep. 15, 2006. The entire content of the prior application is incorporated herein by reference.

### TECHNICAL FIELD

This invention relates to assays for detecting the native-state of a protein and screening methods to identify compounds that modulate the stability of the native-state of a protein.

### BACKGROUND

Maintaining the native-state of a protein (proper assembly and folding of a protein) is a complex process and dysregulation of this process can carry significant pathological consequences. Numerous diseases are associated with a disruption(s) of the native-state of protein. For example, mutations or deletions in the homo-tetrameric p53 tumor suppressor that prevent its homotetramerization are associated with the development of certain cancers (Soussi T et al. (2001) Nat. Rev. Cancer 3:233-240; and Varley et al. (1997) Brit. J. Cancer 76:1-14). Misfolding of certain oligomer-forming polypeptides can also result in disease. Misfolding of the microtubule-associated protein Tau, for example, is a contributing factor to numerous neurodegenerative diseases including Alzheimer's disease (Johnson et al. (2004) J. Cell Sci. 117:5721-5729; and Kar et al. (2005) Alzheimer Dis. Assoc. Disord. 19(1):S29-S36). In addition, gain or loss-of-function mutations that affect protein folding in the endoplasmic reticulum can also manifest in profound effects on organismal viability. Defects in alpha-galactosidase A, beta-glucocerebrosidase (beta-glucosidase), or alpha-glucosidase can give rise to lysosomal storage diseases such as Fabry disease, Gaucher diseases, and Pompe disease respectively (Sawkar et al. (2002) Proc. Natl. Acad. Sci. USA 99(24):15428-15433; Ulloa-Aguirre et al. (2004) Traffic 5(11):821-837).

Transthyretin (TTR), an oligomer-forming polypeptide also known as prealbumin, is a self-associating, 55 kDa homotetrameric protein complex involved in the systemic transport of thyroxin (T4) and retinol (Damas et al. (2000) J Struct. Biol. 130(2-3):290-299 and Schussler G. C. (2000) Thyroid 10(2):141-149). Transthyretin protein is produced in liver and choroid plexus and secreted into serum and cerebrospinal fluid, respectively (Schussler G. C. (2000) Thyroid 10(2):141-149 and Schreiber G. (2002) Clin. Chem. Lab Med. 40(12):1200-1210). The transthyretin tetramer contains two conical-shaped cavities, symmetric along its structural C₂-axis, which bind two T4 molecules (Foss et al. (2005) Biochemistry 44:15525-15533; Monoco et al. (1995) Science 268(5213):1039-1041; Enegvist et al. J. Biol. Chem. (2004) 279(25):26411-26416; and Green et al. (2005) Proc. Natl. Acad. Sci. USA 102(41):14545-14550). However, less than 1% of transthyretin complexes are occupied with T4 at homeostasis.

Inherited mutations in transthyretin are causative defects for both familial amyloid polyneuropathy (FAP) and familial amyloid cardiomyopathy (FAC). Disease results from neurodegeneration and/or organ dysfunction associated with transthyretin amyloid fibril deposits in a variety of tissues, particularly the peripheral and central nervous system and heart. There are now over 80 point mutations in transthyretin associated with familial amyloid disease (Foss et al. (2005) Biochemistry 44:15525-15533; Lashuel et al. (1999) Biochemistry 38:13560-13573; Westermark et al. (1990) Proc. Natl. Acad. Sci. USA 87:2843-2845; and Moses et al. (1990) J. Clin. Invest. 86:2025-2033), though Senile Systemic Amylodosis (SSA), another transthyretin-related amyloid condition, results from the aggregation of normal transthyretin in ˜25% of individuals over 80 years old. Treatment options for transthyretin-associated familial amyloidosis include surgical removal of fibril deposits and in some cases liver transplant. The latter is a gene therapy approach introducing a wild-type gene into the patient. The effectiveness of transplantation in treating familial amyloid disease is limited by continued production of mutant transthyretin by the choroid plexus. Transplant options are non-viable for SSA patients, since wild-type transthyretin fibrils are deposited.

Transthyretin aggregation occurs through a multi-step process. The first, rate-limiting step, involves the dissociation of tetramer into monomers. In subsequent steps, monomeric transthyretin misfolds (Foss et al. (2005) Biochemistry 44:15525-15533), and unfolded monomers form fibrils and amyloid. The V30M point mutation, the most prevalent in FAP, has been shown by X-ray crystallographic studies to affect 1-sheet folding in the transthyretin monomer by 1 angstrom, which distorts the T4-binding cavity (Foss et al. (2005) Biochemistry 44:15525-15533 and Green et al. (2005) Proc. Natl. Acad. Sci. USA 102(41): 14545-14550). Since the transthyretin tetramer is stabilized by ligand binding, this provides one rationale for small molecule-based therapies in the treatment of transthyretin amyloid diseases.

### SUMMARY

This invention is based, at least in part, on the discovery that treatment of a native-state protein with a denaturant (and optionally a cross-linking agent) allows for the native-state and non-native-state forms of the protein to be distinguished using an immuno-turbidity assay. This discovery led to the development of methods of detecting the presence or amount of stabilized native-state protein (e.g., stabilized tetrameric transthyretin) in a sample. The discovery also led to the development of methods to identify compounds that modulate (e.g., stabilize or destabilize) the native-state of a protein in the presence of a denaturant.

Provided herein is a method of detecting the presence or amount of a stabilized native-state protein in a sample. The method includes the steps of: contacting a sample comprising a protein with a denaturant for a time sufficient to induce conversion of unstabilized native-state protein to a non-native-state of the protein, resulting in mixture (a); contacting mixture (a) with a cross-linking agent for a time sufficient to cross-link the protein, resulting in mixture (b); contacting mixture (b) with an antibody that specifically binds to the protein to form mixture (c), wherein (i) the antibody preferentially binds to cross-linked native-state protein as compared to cross-linked non-native-state protein and binding of the antibody to cross-linked native-state protein results in the formation of insoluble aggregates of the antibody and cross-linked native-state protein, or (ii) the antibody preferentially binds to cross-linked non-native-state protein as compared to cross-linked native-state protein and binding of the antibody to cross-linked non-native-state protein results in the formation of insoluble aggregates of the antibody and cross-linked non-native-state protein; and detecting the presence or amount of insoluble aggregates in mixture (c), wherein detection of insoluble aggregates indicates directly or indirectly the presence or amount of stabilized native-state protein in the sample.

Also provided is a method of detecting the presence or amount of a stabilized native-state protein in a sample that includes the steps of: contacting a sample comprising a protein with a denaturant for a time sufficient to induce conversion of unstabilized native-state protein to a non-native-state of the protein, resulting in mixture (a); contacting mixture (a) with an antibody that specifically binds to the protein to form mixture (b), wherein (i) the antibody preferentially binds to native-state protein as compared to non-native-state protein and binding of the antibody to native-state protein results in the formation of insoluble aggregates of the antibody and native-state protein, or (ii) the antibody preferentially binds non-native state protein and binding of the antibody to non-native-state protein results in the formation of insoluble aggregates of the antibody and non-native-state protein; and detecting the presence or amount of insoluble aggregates in mixture (b), wherein detection of insoluble aggregates indicates directly or indirectly the presence or amount of stabilized native-state protein in the sample. In some embodiments of the method, mixture (a) can be contacted with a diluent prior to contacting mixture (a) with the antibody.

In some embodiments of any of the methods described above, the sample can contain a candidate compound.

In some embodiments of the methods described above, the detection of insoluble aggregates can be a direct indication of the presence or amount of stabilized native-state protein in the sample. In other embodiments of the method described above, the detection of insoluble aggregates can be an indirect indication of the presence or amount of stabilized native-state protein in the sample, the presence or amount of stabilized native-state protein in the sample being inversely proportional to the insoluble aggregates detected.

Featured herein is a method of identifying a compound that stabilizes the native-state of a protein. The method includes the steps of: contacting a sample comprising a protein and a candidate compound with a denaturant for a time sufficient to induce conversion of unstabilized native-state protein to a non-native-state of the protein, resulting in mixture (a); contacting mixture (a) with a cross-linking agent for a time sufficient to cross-link the protein, resulting in mixture (b); contacting mixture (b) with an antibody that specifically binds to the protein to form mixture (c), wherein (i) the antibody preferentially binds to cross-linked native-state protein as compared to cross-linked non-native-state protein and binding of the antibody to cross-linked native-state protein results in the formation of insoluble aggregates of the antibody and cross-linked native-state protein, or (ii) the antibody preferentially binds to cross-linked non-native-state protein as compared to cross-linked native-state protein and binding of the antibody to cross-linked non-native-state protein results in the formation of insoluble aggregates of the antibody and cross-linked non-native-state protein; and detecting the presence or amount of insoluble aggregates in mixture (c), wherein (i) an increase in the formation of insoluble aggregates of the antibody and cross-linked native-state protein in the presence of the candidate compound as compared to in the absence of the candidate compound indicates that the candidate compound stabilizes the native-state of the protein, or (ii) a decrease in the formation of insoluble aggregates of the antibody and cross-linked non-native state protein in the presence of the candidate compound as compared to in the absence of the candidate compound indicates that the candidate compound stabilizes the native-state of the protein. In some embodiments, the protein can be contacted with the candidate compound in vitro or in vivo.

Also featured is a method of identifying a compound that stabilizes the native-state of a protein that includes the steps of: contacting a sample comprising a protein and a candidate compound with a denaturant for a time sufficient to induce conversion of unstabilized native-state protein to a non-native-state of the protein, resulting in mixture (a); contacting mixture (a) with an antibody that specifically binds to the protein to form mixture (b), wherein (i) the antibody preferentially binds to native-state protein as compared to non-native-state protein and binding of the antibody to native-state protein results in the formation of insoluble aggregates of the antibody and native-state protein, or (ii) the antibody preferentially binds non-native state protein and binding of the antibody to non-native-state protein results in the formation of insoluble aggregates of the antibody and non-native-state protein; and detecting the presence or amount of insoluble aggregates in mixture (b), wherein (i) an increase in the formation of insoluble aggregates of the antibody and native-state protein in the presence of the candidate compound as compared to in the absence of the candidate compound indicates that the candidate compound stabilizes the native state of the protein, or (ii) a decrease in the formation of insoluble aggregates of the antibody and non-native-state protein in the presence of the candidate compound as compared to in the absence of the candidate compound indicates that the candidate compound stabilizes the native state of the protein. In some embodiments of the method, mixture (a) can be contacted with a diluent prior to contacting mixture (a) with the antibody. In some embodiments, the protein can be contacted with the candidate compound in vitro or in vivo.

Any of the methods described above can further include the step of measuring the amount of insoluble aggregates of (i) the antibody and native-state protein or (ii) antibody and non-native-state protein.

In some embodiments of any of the methods described herein, the protein can be transthyretin. Transthyretin can be wild-type, mature (processed) transthyretin (i.e., SEQ ID NO:2) or an amyloidogenic form of transthyretin. The amyloidogenic form of transthyretin can be the V30M mutant form of transthyretin (i.e., a processed, human transthyretin mutant polypeptide having the amino acid sequence: GPTGTGESKCPLMVKVLDAVRGSPAINVAMHVFRKAADDTWEPFASGKTSESG ELHGLTTEEEFVEGIYKVEIDTKSYWKALGISPFHEHAEVVFTANDSGPRRYTIAA LLSPYSYSTTAVVTNPKE (SEQ ID NO: 3)), the V122I mutant form of transthyretin, the Y78F mutant form of transthyretin, the L55P mutant form of transthyretin, the L55Q mutant form of transthyretin, the A25T mutant form of transthyretin, the C10R mutant form of transthyretin, the L12P mutant form of transthyretin, the D18E mutant form of transthyretin, the D18G mutant form of transthyretin, the V20I mutant form of transthyretin, the S23N mutant form of transthyretin, the P24S mutant form of transthyretin, the V28M mutant form of transthyretin, the V30A mutant form of transthyretin, the V30L mutant form of transthyretin, the V30L mutant form of transthyretin, the V30G mutant form of transthyretin, the F33I mutant form of transthyretin, the F33L mutant form of transthyretin, the F33V mutant form of transthyretin, the R34T mutant form of transthyretin, the L35N mutant form of transthyretin, the A36P mutant form of transthyretin, the D38A mutant form of transthyretin, the E42G mutant form of transthyretin, the E42D mutant form of transthyretin, the F44S mutant form of transthyretin, the A45D mutant form of transthyretin, the A45S mutant form of transthyretin, the A45T mutant form of transthyretin, the G47R mutant form of transthyretin, the G47A mutant form of transthyretin, the G47V mutant form of transthyretin, the G47E mutant form of transthyretin, the T49A mutant form of transthyretin, the T49I mutant form of transthyretin, the S50R mutant form of transthyretin, the S50I mutant form of transthyretin, the E51G mutant form of transthyretin, the S52P mutant form of transthyretin, the G53E mutant form of transthyretin, the E54G mutant form of transthyretin, the E54K mutant form of transthyretin, the L55R mutant form of transthyretin, the H56R mutant form of transthyretin, the L58H mutant form of transthyretin, the L58R mutant form of transthyretin, the T59K mutant form of transthyretin, the T60A mutant form of transthyretin, the E61K mutant form of transthyretin, the F64L mutant form of transthyretin, the F64S mutant form of transthyretin, the I68L mutant form of transthyretin, the Y69H mutant form of transthyretin, the K70N mutant form of transthyretin, the V71A mutant form of transthyretin, the I73V mutant form of transthyretin, the S77F mutant form of transthyretin, the S77Y mutant form of transthyretin, the I84S mutant form of transthyretin, the I84N mutant form of transthyretin, the I84T mutant form of transthyretin, the E89Q mutant form of transthyretin, the E89K mutant form of transthyretin, the A91S mutant form of transthyretin, the A97G mutant form of transthyretin, the A97S mutant form of transthyretin, the I107V form of transthyretin, the I107M mutant form of transthyretin, the A109S mutant form of transthyretin, the L111M mutant form of transthyretin, S112I mutant form of transthyretin, the Y114C mutant form of transthyretin, the Y114H mutant form of transthyretin, the Y116S mutant form of transthyretin, the A120S mutant form of transthyretin, the Val122 deletion mutant form of transthyretin, or the V122A mutant form of transthyretin (the amino acid numbering system for the aforementioned amyloidogenic mutant forms of transthyretin is based on the amino acid numbering of the mature form of human, wild-type transthyretin (127 amino acids) as depicted in SEQ ID NO:2).

In any of the methods described herein the native-state of transthyretin can be a tetramer. In other embodiments of the methods described herein, the native-state of transthyretin can be a properly-folded monomeric form of transthyretin and the corresponding non-native-state of transthyretin can be a misfolded (or amyloid) form of monomeric transthyretin.

In any of the methods described herein, a human subject (e.g., a human patient) can have, or be at risk of developing (e.g., have a genetic predisposition to developing), a transthyretin amyloid disease. As used herein, a “transthyretin amyloid disease” is any pathology or disease associated with dysfunction or dysregulation of transthyretin that results in formation of transthyretin-containing amyloid fibrils. Dysregulation or dysfunction of a transthyretin polypeptide includes an enhanced or increased propensity (e.g., by virtue of mutation of one or both copies of the transthyretin gene) of tetrameric transthyretin to disassociate into monomers. Dysregulation or dysfunction of transthyretin can also include an increased propensity of transthyretin monomers to denature (misfold) or otherwise misassemble into amyloid or other abnormal quaternary structures associated with transthyretin amyloid diseases. The misfolding and/or aggregation of a transthyretin can be misfolding or aggregation of a wild-type or mutant form of transthyretin. Mutant forms of transthyretin polypeptides include, for example, any of the mutant forms or mutations described herein. Mutations in a transthyretin gene can also lead to, for example, dysregulated expression of a transthyretin molecule (e.g., overexpression of transthyretin). Overexpression or disease-causing expression of transthyretin can be overexpression of a mutant form of transthyretin or a wild-type transthyretin polypeptide. Transthyretin amyloid diseases include, but are not limited to, familial amyloid polyneuropathy, familial amyloid cardiomyopathy, senile systemic amyloidosis, cardiac amyloidosis following liver transplantation, peripheral nerve amyloidosis following liver transplantation, leptomeningeal amyloidosis, transthyretin mutant-associated carpal tunnel syndrome, vitreous deposition, or transthyretin mutant-associated skin amyloidosis.

In any of the methods described herein, the protein can be an amyloidogenic protein such as beta-amyloid, alpha-synuclein, tau, an immunoglobulin light chain, serum amyloid A protein, serum amyloid P protein, apoA-I, beta2-microglobulin, gelsolin, a lysozyme, insulin, fibrinogen, a prion protein, superoxide dismutase, calcitonin, cystatin C, and atrial natriuretic peptide. Prion proteins include, for example, scrapie prion, transmissible mink encephalopathy prion, chronic wasting disease prion, bovine spongiform encephalopathy prion, feline spongiform encephalopathy, exotic ungulate encephalopathy prion, Kuru prion, Creutzfeldt-Jakob disease prion, variant Creutzfeldt-Jakob disease prion, Gerstmann-Staussler-Scheinker syndrome prion, or fatal familial insomnia prion.

In any of the methods described herein, the protein can be a trafficking-defective protein. The trafficking defective protein can be a wild-type form of the protein (e.g., overexpression of a non-mutant form of a protein such as alpha-glucosidase) or can be a mutant form of a trafficking-defective protein. Trafficking-defective proteins can include, but are not limited to, gonadotropin-releasing hormone receptor, water-channel aquaporin-2, alpha1-antitrypsin, alpha1-antitrypsin variant, alpha-subunit of hNav1.5, nephrin, multi-drug resistance protein 2, the PHEX gene product, pendrin, sulfonylurea receptor 1, AE1, ceruloplasmin, palmitoyl protein thioesterase 1, cartilage oligomeric matrix protein, the ELOVL4 gene product, aspartyl-glucosaminidase, connexin 32, rhodopsin, cystic fibrosis transmembrane conductance regulator protein, HFE, tyrosinase, protein C, complement C1 inhibitor, alpha-D-galactosidase, beta-hexosaminidase, sucrase-isomaltase, UDP-glucuronosyl-transferase, insulin receptor, growth hormone receptor, myeloperoxidase (MPO), preproparathyroid hormone, Human Ether-a-go-go-Related gene (HERG), tyroxine binding globulin, lipoprotein lipase, low-density lipoprotein receptor, microsomal triglyceride transfer protein, apolipoprotein a, thyroglobulin, type I pro-collagen, fibrinogen, alpha 1-antichymotrypsin (ACT), phosphoinositide-dependent kinase 1, phosphoinositide-dependent kinase 2, vasopressin precursor protein prepro-vasopressin neurophysin II, peripheral myelin protein 22, proteolipid protein, presenilin, von Willebrand factor, ERGIC-53/p58, or sedlin. Common trafficking-defective mutations in cystic fibrosis transmembrane conductance regulator protein include, for example, the AF508 mutant form of the cystic fibrosis transmembrane conductance regulator protein. Mutations of beta-glucosidase can include the N370S mutant of beta-glucosidase. Further examples of trafficking-defective mutations giving rise to diseases such as Fabry disease and glycogen storage disease type II can be found, for example, in Eng et al. (1993) Am. J. Hum. Genet. 53(6):1186-1197 and Hermans et al. (2004) Hum. Mut. 23(1):47-56.

In other embodiments of any of the methods described herein, the native-state of a protein can be an oligomeric-form of a protein. Oligomer-forming polypeptides useful in any of the methods described herein include Alpha-Beta protein, superoxide dismutase, Abri/ADan, glial fibrillary acidic protein, ATP7B, hemoglobin, amyloid A, beta-2-microglobulin, custatin C, lysozyme, fibrinogen, AH and AL immunoglobulin proteins, ApoAI, ApoAII, gelsolin, lactoferrin, lactohedrin, survivin, EGF-R, Erb-B2, or IL-12.

In any of the embodiments of the methods described herein, the sample can be provided or obtained from a mammal (e.g., a human (e.g., a human patient) or a primate (e.g., a chimpanzee, a baboon, or a monkey), a mouse, a rat, a rabbit, a guinea pig, a gerbil, a hamster, a horse, any one of a livestock (e.g., a cow, a pig, a sheep, or a goat), a dog, a cat, or a whale.

In any of the methods described herein, a human subject can have, or be at risk of developing, Alzheimer's disease, Dutch cerebrovascular amyloidosis, Flemish cerebrovascular amyloidosis, Italian cerebrovascular amyloidosis, progressive supranuclear palsy, progressive subcortical gliosis, Pick's disease, dementia pugilisticas, Parkinson's Disease, kuru, Creutzfeldt-Jakob disease, Alexander Disease, Wilson Disease, Lou Gehrig's Disease, sickle cell anemia, cystic fibrosis, diabetes, and Huntington's disease.

In some embodiments of any of the methods described herein, a human subject can have, or be at risk of developing, a lysosomal storage disease. As used herein, a “lysosomal storage disease” is any disorder or pathology that in many cases is caused by genetic defects that affect one or more lysosomal enzymes. Lysosomal storage diseases result from a deficiency in a particular protein (e.g., enzyme) activity present in the lysosomal compartment. Deficiencies of particular protein activities can be the result of lowered or absent expression of the protein or can be the result of one or more mutations in the protein. One or more mutations of the protein can affect the activity of the protein in a number of ways. Mutations can occur in the active site of the protein (e.g., the active site of an enzyme (e.g., the kinase domain of a kinase)). Mutations can also be mutations that destabilize the native-state of a protein. In some embodiments, the one or more mutations of a protein can impair the trafficking of the protein through the endoplasmic reticulum as described above. Examples of lysosomal storage diseases include, but are not limited to, Fabry disease, Gaucher disease (type 1, type 2, or type 3), Pompe disease, Hurler/Scheie syndrome, MPS type I, GM1gangliosidosis, galactosialidosis, Morquio syndrome B, MPS type IVB, Sandhoff disease, Tay-Sachs disease, beta-mannosidosis, alpha-L-fucosidosis, Maroteaux-Lamy syndrome, MPS type VI, metachromatic leukodystrophy, Schindler disease, aspartylglycosaminuria, Hunter syndrome, MPS type II, Sanfilippo syndrome A, MPS type IIIA, Sanfilippo syndrome B, MPS type IIIB, Sanfillipo syndrome C, MPS type IIIC, Sanfilippo syndrome D, MPS type IID, Morquio syndrome A, MPS type IVA, Sly syndrome, MPS type VII, hyaluronidase deficiency, MPS type IX, multiple sulfatase deficiency, alpha-mannosidosis, sialidosis, X-linked ictiosis and multiple sulfatase deficiency, mucolipidodisis II, mucolipidosis III, Wolman disease, Farber disease, Niemann-Pick disease A, Niemann-Pick disease B, glycogenosis type II, neuronal ceroid lipofucsinosis infantile type, neuronal ceroid lipofucsinosis late infantile type, neuronal ceroid lipofucsinosis juvenile type, Krabbe disease, lysosomal acid phosphatase deficiency, pycnodysostosis, cystinosis, sialic acid storage disease, cobalamin deficiency type F, Niemann-Pick disease type C, galactosialidosis, metachromatic leukodystrophy variant, Gaucher disease variant, Tay-Sachs disease type AB, or glycogen storage disease.

Alternatively, the invention also features methods for detecting the non-native-state of the protein. The method includes the steps of: contacting a sample comprising a protein with a denaturant for a time sufficient to induce conversion of unstabilized native-state protein to a non-native-state of the protein, resulting in mixture (a); contacting mixture (a) with a cross-linking agent for a time sufficient to cross-link the protein, resulting in mixture (b); contacting mixture (b) with an antibody that specifically binds to the protein to form mixture (c), wherein (i) the antibody preferentially binds to cross-linked native-state protein as compared to cross-linked non-native-state protein and binding of the antibody to cross-linked native-state protein results in the formation of insoluble aggregates of the antibody and cross-linked native-state protein, or (ii) the antibody preferentially binds to cross-linked non-native-state protein as compared to cross-linked native-state protein and binding of the antibody to cross-linked non-native-state protein results in the formation of insoluble aggregates of the antibody and cross-linked non-native-state protein; and detecting the presence or amount of insoluble aggregates in mixture (c), wherein detection of insoluble aggregates indicates directly or indirectly the presence or amount of non-native-state protein in the sample. In another embodiment, the method includes the steps of: contacting a sample comprising a protein with a denaturant for a time sufficient to induce conversion of unstabilized native-state protein to a non-native-state of the protein, resulting in mixture (a); contacting mixture (a) with an antibody that specifically binds to the protein to form mixture (b), wherein (i) the antibody preferentially binds to native-state protein as compared to non-native-state protein and binding of the antibody to native-state protein results in the formation of insoluble aggregates of the antibody and native-state protein, or (ii) the antibody preferentially binds non-native state protein and binding of the antibody to non-native-state protein results in the formation of insoluble aggregates of the antibody and non-native-state protein; and detecting the presence or amount of insoluble aggregates in mixture (b), wherein detection of insoluble aggregates indicates directly or indirectly the presence or amount of non-native-state protein in the sample. In some embodiments of the method, mixture (a) can be contacted with a diluent prior to contacting mixture (a) with the antibody. In some embodiments of any of the methods of detecting non-native-state protein, the sample can contain a candidate compound.

The invention also provides a method of identifying a compound that destabilizes the native-state of a protein that includes the steps of: contacting a sample comprising a protein and a candidate compound with a denaturant for a time sufficient to induce conversion of unstabilized native-state protein to a non-native-state of the protein, resulting in mixture (a); contacting mixture (a) with a cross-linking agent for a time sufficient to cross-link the protein, resulting in mixture (b); contacting mixture (b) with an antibody that specifically binds to the protein to form mixture (c), wherein (i) the antibody preferentially binds to cross-linked native-state protein as compared to cross-linked non-native-state protein and binding of the antibody to cross-linked native-state protein results in the formation of insoluble aggregates of the antibody and cross-linked native-state protein, or (ii) the antibody preferentially binds to cross-linked non-native-state protein as compared to cross-linked native-state protein and binding of the antibody to cross-linked non-native-state protein results in the formation of insoluble aggregates of the antibody and cross-linked non-native-state protein; and detecting the presence or amount of insoluble aggregates in mixture (c), wherein (i) a decrease in the formation of insoluble aggregates of the antibody and cross-linked native-state protein in the presence of the candidate compound as compared to in the absence of the candidate compound indicates that the candidate compound destabilizes the native-state of the protein, or (ii) an increase in the formation of insoluble aggregates of the antibody and cross-linked non-native state protein in the presence of the candidate compound as compared to in the absence of the candidate compound indicates that the candidate compound destabilizes the native-state of the protein. In some embodiments, the protein can be contacted with the candidate compound in vitro or in vivo. In some embodiments of the method, the protein can be a protein whose native-state causes a disease, e.g., an oncogene. The oncogene can be, for example, ras, myc, HER-2/neu (erB-2), hTERT, Bcl-2, src, raf, Bruton's agammaglobulinemia tyrosine kinase (BTK), platelet-derived growth factor receptor, vascular endothelial growth factor receptor, epidermal growth factor receptor, and aurora kinases. The protein can also be a viral protein such as, but not limited to, any of the viral proteins described herein.

Also featured is a method of identifying a compound that destabilizes the native-state of a protein that includes the steps of: contacting a sample comprising a protein and a candidate compound with a denaturant for a time sufficient to induce conversion of unstabilized native-state protein to a non-native-state of the protein, resulting in mixture (a); contacting mixture (a) with an antibody that specifically binds to the protein to form mixture (b), wherein (i) the antibody preferentially binds to native-state protein as compared to non-native-state protein and binding of the antibody to native-state protein results in the formation of insoluble aggregates of the antibody and native-state protein, or (ii) the antibody preferentially binds non-native state protein and binding of the antibody to non-native-state protein results in the formation of insoluble aggregates of the antibody and non-native-state protein; and detecting the presence or amount of insoluble aggregates in mixture (b), wherein (i) a decrease in the formation of insoluble aggregates of the antibody and native-state protein in the presence of the candidate compound as compared to in the absence of the candidate compound indicates that the candidate compound destabilizes the native state of the protein, or (ii) an increase in the formation of insoluble aggregates of the antibody and non-native-state protein in the presence of the candidate compound as compared to in the absence of the candidate compound indicates that the candidate compound destabilizes the native state of the protein. In some embodiments of the method, mixture (a) can be contacted with a diluent prior to contacting mixture (a) with the antibody. In some embodiments, the protein can be contacted with the candidate compound in vitro or in vivo. In some embodiments of the method, the protein can be a protein whose native-state causes a disease, e.g., an oncogene. The oncogene can be, for example, any oncogene described herein. The protein can also be a viral protein such as, but not limited to, any of the viral proteins described herein.

In some embodiments of any of the methods described herein, the sample can be, or can contain, blood, plasma, serum, urine, semen, lachrymal fluid, cerebrospinal fluid, vitreous humor, or a tissue sample.

The denaturant in any of the methods described herein can be, for example, urea, guanadinium hydrochloride, guanadinium sulfite, guanadinium thiocyanate, sodium n-dodecyl sulfate, Nonindet-40 (NP40), n-lauryl sarcosine, high or low pH (e.g., base or acid treatment), or heat. The cross-linking agent in any of the methods described herein can be glutaraldehyde, succinimidyl acetylthioacetate (SATA), ethylene glycol disuccinate di-(N-succinimidyl) ester (EGS), isocyanate, ultra-violet light, bis-(maleimideo)-methyl ether (BMME), or carbodiimide 1-ethyl-3-(3-dimethyl amino propyl) carbodiimide hydrochloride (EDC).

Provided herein is a kit for detecting the presence or amount of stabilized native-state protein. The kit contains: a denaturant; a cross-linking agent; an antibody specific for the protein; instructions for use in detection of stabilized native-state protein, and optionally instructions for use in measuring the presence or amount of stabilized native-state protein.

Also featured is a kit for detecting the presence or amount of stabilized native-state protein containing: a denaturant; an antibody specific for the protein that preferentially binds to native-state protein as compared to non-native-state protein; instructions for use in detection of stabilized native-state protein, and optionally instructions for use in measuring the presence or amount of stabilized native-state protein.

Any of the kits described herein can contain any of the denaturants (e.g., urea) or cross-linking agents (e.g., glutaraldehyde) described herein.

In some embodiments of any of the kits described herein, the protein can be transthyretin and the native-state of transthyretin can be tetrameric transthyretin.

“Polypeptide” and “protein” are used interchangeably and mean any peptide-linked chain of amino acids, regardless of length or post-translational modification. The proteins used in any of the methods of the invention can contain or be wild-type proteins or can be variants that have not more than 50 (e.g., not more than one, two, three, four, five, six, seven, eight, nine, 10, 12, 15, 20, 25, 30, 35, 40, or 50) conservative amino acid substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. All that is required is that such variants of oligomer-forming polypeptides have at least 25% (e.g., at least: 30%; 40%; 50%; 60%; 70%; 75%; 80%; 85%; 90%; 95%; 97%; 98%; 99%; 99.5%, or 100% or even greater) of the ability of the wild-type, full-length, mature oligomer-forming polypeptide from which they were derived to assemble into oligomeric forms.

A “polypeptide fragment,” “protein fragment” as used herein, refers to a segment of the polypeptide that is shorter than a full-length, unprocessed polypeptide. A “functional fragment” of a protein is a fragment of the protein that still retains the ability of the full-length, mature, wild-type protein to assume its native-state. Fragments of a polypeptide include terminal as well internal deletion variants of a polypeptide. Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids.

As used herein, the terms “native-state” or “native-state of a protein” refer to the properly folded or assembled form of a protein. In most cases, the native-state of a protein is the operable or functional form of the protein. The native-state of the protein includes the natural or physiologic form of the protein that occurs in a wild-type cell or whole organism, the cell or whole organism being unaffected by any exogenous factors that directly or indirectly affect the stability of the polypeptide. Where the protein is a monomeric protein or exists as a monomeric form the protein (e.g., a form of the protein that includes only one polypeptide), the “native-state” of the protein indicates a properly folded form of the monomeric protein as compared to a denatured, unfolded, or improperly folded form of the monomeric protein. Where the operable or functional form of a protein is an oligomeric form of a protein (e.g., a homodimer, a homotrimer, a homotetramer, a heterodimer, a heterotrimer, or a heterotetramer), the native-state of the protein includes the oligomeric form of the protein. For example, the operable or functional physiologic form (i.e., the native-state) of transthyretin is a homo-tetrameric form of transthyretin. In some instances, a protein (e.g., certain enzymes or other non-structural proteins) can have more than one native-state where the protein operates or undergoes regulation by conformational transitioning between these one or more native-states. In addition, in some instances a protein can have an oligomeric native-state (e.g., a transthyretin tetramer) and a properly-folded monomeric native-state (e.g., a properly-folded transthyretin monomer), where the native-state monomeric form is properly folded form that can denature or misfold into a non-native-state of the native-state monomer. As used herein, a “non-native-state” of a polypeptide, as compared to the “native-state” of the protein, is a form of the protein that is not operable or functional or is a form of the protein that is an unfolded, denatured, or otherwise improperly-folded (e.g., an amyloid form of transthyretin or tau).

As used herein, a “trafficking-defective” protein is a protein that fails to properly transit (e.g., fails to properly fold and/or oligomerize where the protein is an oligomer-forming polypeptide) the endoplasmic reticulum. Typically a protein that fails to properly transit the endoplasmic reticulum is a protein that has misfolded during its biosynthesis or processing in the endoplasmic reticulum. Misfolding can result from one or more mutations in a protein that destabilize the native-state of the protein. Misfolding can also result from heightened or overexpression of a wild-type form (or mutant) of the protein. Other factors that may trigger protein misfolding include aberrant temperature, oxidative stress, activation of signaling pathways associated with protein folding and quality control, or lack of chaperone constituents that are necessary to help guide proper conformation of a protein.

As used herein, an “oligomer” or “oligomeric form” of a polypeptide is a form of the polypeptide that consists of two or more (e.g., two, three, four, five, six, seven, eight, nine, ten, or up to 20) monomeric forms of the polypeptide. Where the oligomeric forms of the oligomer-forming polypeptide are composed of only one oligomer-forming polypeptide, the oligomer is a homo-oligomer. Where the oligomeric form of a polypeptide is an oligomeric form composed of one or more (e.g., one, two, three, four, five, six, seven, eight, nine, ten, or up to 20) monomers of at least two (e.g., at least two, at least three, at least four, at least five, at least six, at least seven, at least ten) different polypeptides, the oligomers are hetero-oligomers (e.g., heterodimers of two proteins). Oligomeric forms can include forms where the polypeptide monomers are covalently joined together (e.g., covalently joined by disulfide linkage), or can include oligomeric forms that are joined non-covalently based on protein-protein interactions (e.g., through a protein dimerization domain (e.g., the dimerization domain of a transthyretin polypeptide).

An “oligomer-forming polypeptide” refers to any polypeptide that is capable of assembling into an oligomeric form. The oligomer-forming polypeptide can be a polypeptide which naturally self-assembles into an oligomer, for example, transthyretin that self-assembles into a homo-tetramer. The oligomer-forming polypeptide can also be a polypeptide that is artificially made to oligomerize, e.g., a polypeptide subjected to certain experimental conditions, or modified in such ways, that promote a non-physiological assembly of the polypeptide (e.g., a fusion polypeptide of a non-oligomer-forming polypeptide and a polypeptide or fragment of an oligomer-forming polypeptide (e.g., a glutathione-S-transferase fusion with a non-oligomer-forming polypeptide)).

Transthyretin, as used herein, contains, or is: (a) a full-length, wild-type human transthyretin molecule (SEQ ID NO:1); (b) a mature, processed form of the wild-type human transthyretin molecule (SEQ ID NO:2); (c) a disease-associated or “amyloidogenic” mutant of transthyretin; (d) a functional fragment of (a), (b), or (c); or (e) any one of (a), (b), (c), or (d) with not more than 50 (see above) conservative substitutions. Disease-associated or amyloidogenic mutant variants of transthyretin include, but are not limited to, any of the amyloidogenic mutant forms of transthyretin described herein. Transthyretin can also be from any species (e.g., bird, reptile, or mammal (e.g., a mouse, rat, dog, cat, goat, pig, cow, horse, whale, or monkey)) that expresses a homolog of a human transthyretin polypeptide (SEQ ID NO:1). “Functional fragments” of a transthyretin include fragments that (a) comprise at least 20 (e.g., at least 30, 40, 50, 60, 70, 80, 90, 100, 110, 120, 130, or 140) contiguous amino acids of wild-type transthyretin, but are (b) shorter than the full-length transthyretin polypeptide by at least one amino acid (e.g., at least one amino acid, at least two amino acids, at least three amino acids, at least 5 amino acids, at least 10 amino acids, at least 15 amino acids, at least 20 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 80 amino acids, at least 90 amino acids, at least 95 amino acids, at least 100 amino acids, at least 105 amino acids, at least 110 amino acids, at least 120 amino acids, or at least 125 amino acids) and that still retain the ability to oligomerize (e.g., form tetramers).

As used herein, the term “stabilized” (in the context of the native-state of a protein such as transthyretin) refers to the effect of a stabilizing agent (e.g., a test compound or a candidate therapeutic agent) in preventing or reducing conversion of native-state of a protein to a non-native-state of the protein in the presence of a denaturant (e.g., urea). Thus, a transthyretin tetramer, for example, is said to be “stabilized” by a stabilizing agent when one or more of the transthyretin tetramers in a sample are preserved in the presence of a denaturant. Likewise, “unstabilized” (e.g., “unstabilized transthyretin tetramer”) refers to native-state protein that undergoes partial or complete conversion to non-native-state protein in the presence of a denaturant. Unstabilized transthyretin tetramer, for example, includes transthyretin tetramer not exposed to a stabilizing agent (e.g., a negative control in an assay) or to transthyretin tetramer exposed to a test compound that does not stabilize the tetramer. Stabilization of native-state protein by a stabilizing agent can be 100% (i.e., 100% of the native-state protein is refractory to dissociation in the presence of the denaturant) or can be less than 100% (e.g., 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99%). Stability can depend on both (a) the stabilizing agent (e.g., the test compound) used and (b) the limits of detection for the particular assay method.

As used herein, “insoluble aggregate” is a protein complex formed of (i) native-state protein and one or more of an antibody specific for the protein, where the antibody preferentially binds to native-state protein as compared to non-native-state protein (e.g., a transthyretin-specific antibody complexed with tetrameric transthyretin) or (ii) non-native-state protein and one or more of an antibody specific for a protein, where the antibody preferentially binds to non-native-state protein as compared to native-state protein. The binding of one or more of the antibody to native-state or non-native-state protein results in the formation of large complexes that are no longer soluble in the solution from which they were dissolved. Such insoluble aggregates can be detected in a solution using photometric methods as described herein.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can also be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., inhibiting survival of cancer cells, will be apparent from the following description, from the drawings and from the claims.

### DESCRIPTION OF THE DRAWINGS

FIG. 1 is a line graph depicting the concentration of purified, recombinant monomeric (diamonds) and tetrameric (squares) forms of transthyretin (TTR) in solution as determined using the Olympus OSR6175 reagent. The X-axis represents the known, input concentration of the proteins and the Y-axis is the concentration determined by the immuno-turbidimetric assay. The concentrations of the proteins are given in units of milligrams (mg)/deciliter (dL).

FIG. 2 is a line graph showing the effect of glutaraldehyde cross-linking on the detection of purified, recombinant monomeric (squares) and tetrameric forms of transthyretin (TTR) in solution as determined using the Olympus OSR6175 reagent. Samples of purified transthyretin monomer or tetramer were treated with urea buffer (final concentrations: 4.8 M urea, 25 mM sodium phosphate, 50 mM potassium chloride, pH 7.4) followed by the immediate addition of the indicated concentration of glutaraldehyde (0-40 mM final) (X-axis). The samples were analyzed using the Olympus OSR6175 reagent, and the measured concentration of the proteins are given in units of milligrams (mg)/deciliter (dL). The input concentration of transthyretin monomer and tetramer was 10 mg/dL final.

FIG. 3 is a flow chart depicting the steps of the immuno-turbidimetric assay in the presence and absence of the test compound 2-(2,6-dichloro-phenyl)-benzoxazole-6-carboxylic acid (Cmp. 1). DMSO refers to dimethyl sulfoxide and is the solvent in which the compound was dissolved.

FIG. 4A is a flow chart depicting the alternative detection steps of the protein stability assay (western blot and immuno-turbidimetric assay formats).

FIG. 4B is an immunoblot depicting the stability of transthyretin tetramers in plasma samples subjected to urea treatment in the presence and absence of the test compound 2-(2,6-dichloro-phenyl)-benzoxazole-6-carboxylic acid. Plasma samples were solubilized in Laemmli buffer, subjected to sodium-dodecyl sulfate-polyacrylamide gel electrophoresis (SDS-PAGE) followed by western blotting using antibodies specific for transthyretin. The position of transthyretin tetramer on the immunoblot is indicated at the left of the immunoblot, and the time point at which the samples were collected for assay (0 or 3 days) are indicated above the immunoblot.

FIG. 5 is a bar graph depicting the concentration of purified, recombinant monomeric and tetrameric forms of transthyretin as determined by immuno-turbidimetric assay using Beckman reagents in the presence and absence of 4.8 M urea. The concentration of the proteins are expressed in units of mg/dL.

FIG. 6 is a line graph depicting the effect of different concentrations of 2-(3,5-dichloro-phenyl)-benzoxazole-6-carboxylic acid (Compound 2 (Cmp. 2)) on transthyretin tetramer stabilization using immuno-turbidimetric assay 1. The X-axis represents time and is given in days; the Y-axis represents the percentage of tetramer remaining in solution after treatment with urea. Concentrations of Cmp. 2 were 2.2 μM (circles), 4.3 M (cross), 8.6 μM (square), or 0 μM (DMSO control, triangles) as indicated.

FIG. 7 is a line graph depicting the effect of different concentrations of 2-(3,5-dichloro-phenyl)-benzoxazole-6-carboxylic acid (Compound 2 (Cmp. 2)) on transthyretin tetramer stabilization using immuno-turbidimetric assay 2. The X-axis represents time and is given in hours; the Y-axis represents the percentage of tetramer remaining in solution after treatment with urea. Concentrations of Cmp. 2 were 4.6 μM (square), 9.2 M (triangle), or 0 μM (DMSO control, diamond) as indicated.

FIG. 8 is a line graph depicting destabilization kinetics using plasma samples containing different concentrations of tetrameric transthyretin in the presence of 7.2 μM 2-(3,5-dichloro-phenyl)-benzoxazole-6-carboxylic acid (Compound 2 (Cmp. 2)). The X-axis represents time and is given in days; the Y-axis represents the percentage of tetramer remaining in each sample following treatment with urea. Concentrations of tetrameric transthyretin are reported as “high” (˜30.1 mg/dL; diamonds), “med” (medium, ˜25.4 mg/dL; squares), or “low” (˜17.7 mg/dL; triangles).

FIG. 9 is a scatter plot depicting the destabilization kinetics of tetrameric transthyretin using plasma samples from patients harboring the V30M transthyretin mutation. Pooled plasma samples were treated in triplicate with Cmp. 2 (3.6 or 7.2 μM), or DMSO as a control, for 15 minutes prior to the addition of a urea buffer. This experiment was conducted on three separate days, which is indicated by the filled circles (experiment 1), open circles (experiment 2), and open diamonds (experiment 3). The X-axis represents the dose of Cmp. 2 (in M) and the amount of time (in hours) that the samples were subjected to the urea buffer. The Y-axis represents the percentage of the initial (“Fraction of initial”) transthyretin tetramer concentration remaining in each sample following treatment with urea.

### DETAILED DESCRIPTION

### Methods of Detecting Native-State Protein

Featured herein are immuno-turbidimetric methods for detecting the presence or amount of stabilized native-state protein in a sample.

The methods can be useful as a diagnostic test (e.g., a test for a biomarker) for the presence or amount of native-state protein in samples obtained from a subject. Such a method can be useful, for example when the protein is transthyretin, in identifying a subject (e.g., a human patient) as having, or at risk of developing (e.g., having a genetic predisposition to developing), any transthyretin amyloid disease described herein. Where the protein is any one of the amyloidogenic proteins described herein, the method can also be useful in identifying subjects having, or at risk of developing, Alzheimer's disease or Dutch, Flemish, or Italian cerebrovascular amyloidoses (AB protein); progressive supranuclear palsy, progressive subcortical gliosis, Pick's disease, dementia pugilisticas, and other tauopathies (Tau protein); Parkinson's Disease (alpha-synuclein); kuru or Creutzfeldt-Jakob disease (Prion proteins); Alexander Disease (GFAP); Wilson Disease (ATP7B); Lou Gehrig's Disease (SOD-1); sickle cell anemia (hemoglobin); cystic fibrosis (CFTR); diabetes (insulin); or Huntington's disease (polyglutamate) (disease-associated amyloid protein is indicated in parentheses; Howlett D. R. (2003) Curr. Med. Chem.-Immun., Endoc., & Met. Agents 3:371-383; Aridor et al. (2000) Traffic 1:836-851; Aridor et al. (2002) Traffic 3:781-790; and U.S. Patent Publication No. US2004/0166554 A1; all of which are incorporated herein by reference in their entirety). Where the protein is a protein associated with a lysosomal storage disease (e.g., alpha1-galactosidase or alpha-glucosidase), the method can also be used in identifying subjects having, or at risk of developing a lysosomal storage disease such as any of those described herein.

Where the protein of the method is contacted with a compound in vivo, the method can be used to evaluate the effectiveness of a compound, administered to a subject, in stabilizing the native-state (e.g., a transthyretin tetramer) of a protein (e.g., transthyretin). The subject can be a patient having, or at risk of developing, familial amyloid polyneuropathy, familial amyloid cardiomyopathy, senile systemic amyloidosis, cardiac amyloidosis following liver transplantation, peripheral nerve amyloidosis following liver transplantation, leptomeningeal amyloidosis, transthyretin mutant-associated carpal tunnel syndrome, vitreous deposition, or transthyretin mutant-associated skin amyloidosis or any subject having any amyloidogenic mutation in transthyretin (e.g., a subject or patient expressing any amyloidogenic transthyretin described herein (e.g., the V30M mutant form of transthyretin)). The subject can also be a patient having, or at risk of developing, a lysosomal storage disease such as any of the lysosomal storage diseases described herein. The presence or amount of the native-state of a polypeptide (as a measure of the presence or amount of insoluble aggregates) can be assayed in samples taken from a subject before and after administering to the subject a test or candidate compound. In this case, an increase in the presence or amount of stabilized native state protein present in the sample from the subject following administration of the compound as compared to the presence or amount of native-state protein in the sample obtained (i.e., taken or provided) before administration of the compound is an indication that the compound increases the stability of the native-state protein. The subject can alternatively be an animal (e.g., a test animal (e.g., a mouse, a rat, a guinea pig, a rabbit, a dog, a cat, or a monkey used in an animal model or study)) used to evaluate the efficacy, toxicity, and/or pharmacokinetics of a given compound, for example, during clinical development of a compound. A sample obtained or provided from a subject can also be contacted with the compound in vitro.

Where the sample is contacted with a compound in vitro, the method can be used in biochemical validation studies to test or confirm the mechanism of action of a compound and/or the efficacy of a compound to modulate the stability of native-state protein (e.g., to stabilize tetrameric transthyretin). For example, a compound that was first determined to be useful in treating a familial amyloidotic polyneuropathy in an animal model could be evaluated to assess its mechanism of action. The method can generally involve contacting a sample containing a protein in a buffer with the test compound. Following the denaturation step of the method, the stability of the native-state protein can be evaluated in the presence and absence of the test compound. In this case, an increase in native-state protein present in the sample contacted with the compound as compared to the amount of native-state protein in the absence of the compound, is an indication that the compound increases the stability of the native-state of the protein.

Alternatively, the method can be used in biochemical validation studies to test or confirm the mechanism of action of a compound and/or the efficacy of a compound to destabilize native-state protein (e.g., to destabilize the native-state of an oncogene such as ras or myc). For example, a compound that was first determined to be useful in treating one or more cancer types in animal models could be evaluated to assess its mechanism of action. In this case, a decrease in native-state protein present in the sample contacted with the compound as compared to the amount of native-state protein in the absence of the compound, is an indication that the compound decreases the stability of the native-state of the protein.

The method can also be used generally for scientific research relating to the stability of the native-state of proteins (e.g., transthyretin) or to any other research where monitoring the stability of the native-state of a protein can be useful. The protein can be a protein associated with a disease state (e.g., a human disease such as familial amyloid polyneuropathy) or a protein not directly associated with a disease state, for example, heat shock proteins of bacteria (e.g., GroEL of E. coli).

Examples of immuno-turbidimetric assays are described in, e.g., Liu et al. (2006) Dement. Geriatr. Cogn. Disord. 21:155-161; Bence et al. (2005) Vet. Clin. Pathol. 34(4):335-341; Ledue et al. (2002) Clin Chem Lab Med. 40(5):520-528; Soltys et al. (1998) Blood Purif. 16(3):123-134; and Luo et al. (2004) World J Gastroenterol. 10(9):1292-1296. Such methods can be used for the qualitative or quantitative measurement of drugs or biomarkers (e.g., proteins such as native-state or non-native-state proteins) in bodily fluids such as blood, serum, or plasma. The immuno-turbidimetric assays described herein are based on the preferential binding of an antibody to a native-state or to a non-native-state of a protein. The binding of the antibody to native-state protein, if present in the sample, causes rapid agglutination (aggregation) of the native-state protein and antibody leading to the formation of insoluble aggregates of the antibody and native-state protein in the sample. By linking one or more antibodies to one or more native-state proteins, the antibodies are able to bind many antigen molecules simultaneously. This greatly increases the speed and robustness of the visible reaction (i.e., the immuno-turbidity of the sample). The presence or amount of the insoluble aggregates in the sample can be determined photometrically and is proportional to the difference between incident light delivered to a sample versus the transmitted light emitted from the sample (i.e., the absorbance). The higher the absorbance of a sample, the more insoluble aggregates are present in the sample, which indicates that more native-state protein is present in the sample. Machines or instruments useful in detecting insoluble aggregates are described in the Examples below, and include spectrophotometers such as the Bayer Advia 1650 Chemistry Analyzer (Diamond Diagnostics, Holliston, Mass.), Olympus AU400, Olympus 640, Beckman Synchron CX, Abbott Aeroset, Cobas Mira, Hitachi 704, Hitachi 902, ILAB 600, and Toshiba 80FR NEO. Where the antibody preferentially binds to the non-native-state of the protein, the binding of an antibody to non-native-state protein, if present in the sample, causes rapid agglutination of the non-native-state protein and antibody leading to the formation of insoluble aggregates of the antibody and non-native-state protein in the sample.

Detection of insoluble aggregates can be a direct indication of the presence or amount of stabilized native-state protein in the sample or, where the antibody preferentially binds to non-native-state protein as compared to native-state protein, the detection of insoluble aggregates is an indirect indication of the presence or amount of stabilized native-state protein in the sample. In the latter case, the presence or amount of stabilized native-state protein is inversely proportional to the insoluble aggregates detected (i.e., the more insoluble aggregates of antibody and non-native-state protein, the less stabilized native-state protein in the sample). Methods of assessing the immuno-turbidity of a sample can be quantitative, semi-quantitative, or qualitative. An example of a quantitative determination of the amount of native-state protein is comparing the absorbance of a test sample against the absorbance value obtained from samples of known concentration and thus generating a discreet numerical calculation of the amount of native-state protein in a sample (e.g., in units of mg/dL). A baseline or background for the immuno-turbidimetric assay can be established by detecting or measuring the immuno-turbidity of samples in the absence of the compound, samples containing only the solvent or carrier in which the compound is dissolved (e.g., DMSO), and/or samples containing no protein (native-state protein). The assay can also include a sample or samples that serve as positive controls. As used herein, “positive controls” refer to test samples containing known quantities of reagents (e.g., diluents, cross-linking agents, denaturants, or compounds) and protein (e.g., a known protein concentration of transthyretin) that when assayed by the methods described herein will give a known or expected value. Alternatively, the immuno-turbidity of a sample can be expressed or indicated using a variety of semi-quantitative/qualitative systems known in the art. Thus, the immunoturbidity of a sample can be expressed as, for example, (a) one or more of “excellent”, “good”, “satisfactory”, “unsatisfactory”, and/or “poor”; (b) one or more of “very high”, “high”, “average”, “low”, and/or “very low”; or (c) one or more of “+++++”, “++++”, “+++”, “++”, “+”, “+/−”, and/or “−”.

For any of the methods described herein, the sample can be, for example, any bodily or biological fluid such as blood, plasma, serum, urine, semen, lachrymal fluid, cerebrospinal fluid, or the sample can be a tissue sample. A solid tissue sample can be readily converted to a soluble or semi-soluble form for use in the methods by techniques known to those of skill in the art, including for example, maceration techniques, or passing the tissue through a filter such as cheese-cloth. When the protein, or native-state of a protein, to be tested is in a cell, the sample can also be a lysate prepared from whole cells or cell fractions (e.g., a cell membrane fraction, cell nuclear fraction, or mitochondrial fraction). The sample can optionally be prepared in vitro and contain a purified and/or recombinant protein in an aqueous buffer (optionally with certain salts (e.g., salts of magnesium, manganese, sodium, or potassium) and/or free of other constituents. A buffer can also be used to maintain the protein (e.g., the native-state protein) in a neutral or stable pH, and can include buffer chemicals such as Tris-HCl, HEPES, MOPS, or citrate. The sample can also be free of a protein (either native-state or non-native-state protein), e.g., a negative control for use in the immuno-turbidimetric assays.

Antibodies or antibody fragments that specifically bind to a native-state protein, and useful in the methods described herein, can be generated, for example, by immunization, e.g., using an animal, or by in vitro methods such as phage display. A polypeptide that includes all or part of a given protein can be used to generate an antibody or antibody fragment, for example, mature, full length, wild-type human transthyretin polypeptide with the following amino acid sequence: MASHRLLLLCLAGLVFVSEAGPTGTGESKCPLMVKVLDAVRGSPAINVAVHVFR KAADDTWEPFASGKTSESGELHGLTTEEEFVEGIYKVEIDTKSYWKALGISPFHE HAEVVFTANDSGPRRYTIAALLSPYSYSTTAVVTNPKE (SEQ ID NO: 1). In some embodiments, a portion of the protein (e.g., portion of transthyretin, e.g., with a length of 10, 25, 30, 35, 40, 45, 50, 55, 60, 65, 70, 75, 80, 85, 90, 95, 100, 105, 110, 115, 120, 125, 130, 140, 141, 142, 143, 144, 145, or 146 amino acids; a mature form of transthyretin with the following amino acid sequence: GPTGTGESKCPLMVKVLDAVRGSPAINVAVHVFRKAADDTWEPFASGKTSESGE LHGLTTEEEFVEGIYKVEIDTKSYWKALGISPFHEHAEVVFTANDSGPRRYTIAAL LSPYSYSTTAVVTNPKE (SEQ ID NO: 2)) can be used as an immunogen to generate antibodies that can be screened for reactivity to a native-state or non-native-state protein. In some embodiments, a cell expressing all or part of a protein (e.g., all or part of transthyretin) can be used as an immunogen to generate antibodies.

Methods for testing whether an antibody is specific for a given antigen (i.e., a native-state or non-native state of a protein) such as a transthyretin polypeptide include tests, for example, such as enzyme-linked immunosorbent assays (ELISAs), or immunoblotting techniques such as SDS-PAGE/western blotting, or dot-blot procedures. Specific antibodies generated to an oligomer-forming polypeptide (e.g., an antibody specific for human transthyretin) can be tested for utility in any immuno-turbidimetric assay described herein by routine experimentation and as described in Example 1. Where a positive control antibody exists, i.e., an antibody already known to preferentially bind to a native-state or non-native-state polypeptide and to work in the immuno-turbidimetric assays, the newly generated or test antibody can be used in a parallel immuno-turbidimetric assays and compared against the positive control antibody for efficacy (e.g., the ability to cause the formation of insoluble aggregates in a sample upon preferential binding to native-state or non-native state proteins). While the methods are not limited to any particular reagent, examples of anti-transthyretin antibodies useful in the methods described herein include the anti-transthyretin antibody reagent of the Olympus OSR6175 kit (Olympus, Dublin, Ireland), the anti-transthyretin antibody reagent of the Beckman Prealbumin kit (Prealbumin PAB #475106, Beckman Coulter, Fullerton, Calif.), and the anti-transthyretin antibody reagent of the Bayer Advia reagents (Bayer Healthcare (Diagnostics Group), Tarrytown, N.Y.).

An antibody specific for a protein useful in the methods described herein can be an antibody that preferentially recognizes the native-state of a protein, for example, an antibody that preferentially binds to the tetrameric form of transthyretin (e.g., the anti-transthyretin antibody of the Beckman Prealbumin kit (Prealbumin PAB #475106, Beckman Coulter, Fullerton, Calif.) or the anti-transthyretin antibody of the Bayer Advia reagents). The antibody specific for a protein useful in the methods can also be an antibody that preferentially recognizes a non-native-state of a protein. Antibodies that recognize both the native-state and non-native-state of the protein, for example, an anti-transthyretin antibody that recognizes both tetrameric and monomeric transthyretin (e.g., the anti-transthyretin antibody of the Olympus OSR6175 reagent) can also be useful in the methods described herein. Methods of testing whether an antibody can preferentially bind to the native-state of a protein polypeptide, the non-native-state of a protein, or to both native-state and non-native-state proteins are described in the examples below. Briefly, samples containing only the native-state of a protein (e.g., tetrameric form of transthyretin) or the non-native-state of a protein (e.g., a transthyretin monomer) can be incubated with a candidate antibody and assayed by, for example, immuno-turbidity assay (see Example 1 for determining monomer and tetramer specificity for an anti-transthyretin antibody). Further examples of antibodies exhibiting binding preferences for native-state or non-native-state protein (as compared to the other) are well known to those of skill in the art of molecular biology. For example, numerous antibodies are useful for immunoprecipitation methods (i.e., methods where the protein in its native-state is bound by the antibody) but not for techniques (e.g., western blotting or dot-blotting) where the protein in a non-native-state (e.g., denatured by reducing agents and detergents) is bound by the antibody. Examples of such antibodies with preferences for native-state versus non-native-state protein are described by and commercially available from companies such as BD Biosciences/Pharmingen (Franklin Lakes, N.J.) and Santa Cruz Biotechnologies Inc. (Santa Cruz, Calif.).

The methods provided herein include the step of contacting a sample with a denaturant. As used herein, “denaturant” refers to any agent that induces the conversion of the native-state of a protein to a non-native state of a protein (e.g., causing the native-state of the protein to unfold or an oligomeric, native-state protein to dissociate). Such conversion (denaturation) can, for example, result in some or all of the original properties of the folded native-state protein, especially their biological activity, being diminished or eliminated. Examples of such agents are described herein and generally include, but are not limited to, heat, alkali, or acid. Further examples include: urea, guanadinium hydrochloride, guanadinium sulfite, guandinium thiocyanate, sodium n-dodecyl sulfate, Nonindet-40 (NP40), n-lauryl sarcosine, or other detergents well known to those of skill in the art. The denaturant is generally applied to the sample for a length of time sufficient for the native-state protein to denature. Exemplary concentrations and time duration for denaturation using urea as the denaturant, for example, 1-10M urea (e.g., 1 M urea, 2 M urea, 3 M urea, 4 M urea, 4.8 M urea, 5 M urea, 6 M urea, 7 M urea, 8 M urea, 9 M urea, or 10 M urea) for one or more days (e.g., one day, two days, three days, four days, or five days).

Some of the methods described herein include the step of contacting the sample with a cross-linking agent. As used herein, a “cross-linking agent” is any agent that can covalently join two or more proteins together (e.g., covalently joining two or more transthyretin polypeptides) or covalently join one or more non-adjacent regions within a single protein as predicted from the primary amino acid sequence of the protein. Examples of such cross-linking agents include, for example, UV or X-ray light, chemical cross-linking agents such as glutaraldehyde, succinimidyl acetylthioacetate (SATA), ethylene glycol disuccinate di-(N-succinimidyl) ester (EGS), isocyanate, ultra-violet light, bis-(maleimideo)-methyl ether (BMME), or carbodiimide 1-ethyl-3-(3-dimethyl amino propyl) carbodiimide hydrochloride (EDC). The cross-linking agent is generally applied to the sample for a length of time sufficient for the oligomeric forms of an oligomer-forming polypeptide to be cross-linked, or covalently joined together. Exemplary concentrations and time intervals for cross-linking with glutaraldehyde include approximately 40 mM (e.g., approximately 1 mM, approximately 10 mM, approximately 40 mM, approximately 50 mM, approximately 100 mM, approximately 500 mM, approximately 1 M, or approximately 5 M) for approximately 4 minutes (e.g., approximately 1 minute, approximately 2 minutes, approximately 3 minutes, approximately 4 minutes, approximately 5 minutes, or approximately 6 minutes).

As an alternative to the cross-linking step, the methods described herein can include a step of contacting the sample with a diluent. As used herein, a diluent is any agent that is used to dilute the concentration of any particular compound of a sample or solution, for example, diluting the concentration of a denaturant (e.g., urea) in a sample or a solution. The diluent can be any buffer compatible with the assay, e.g., any aqueous buffer. When only a few (e.g., one, two, three, or four) components of a sample are intended to be diluted, the diluent can optionally contain sufficient concentrations of the other components of the test sample such that the concentration of the other components are not affected by the diluent addition. For example, a diluent containing 50 mM NaCl added 1:1 to a sample containing 50 mM NaCl and 2 M urea would only dilute the urea in the sample. An exemplary dilution ratio for the immuno-turbidimetric assays is approximately 1:5 (e.g., approximately 1:2, approximately 1:3, approximately 1:4, approximately 1:5, approximately 1:6, or approximately 1:10).

### Screening Assays to Identify Compounds that Modulate the Stability of Native-State Protein

Maintaining the native-state of a protein (proper assembly and folding of a protein) is a complex process and dysregulation of this process can often result in disease. Described herein are immuno-turbidimetric screening assay methods for identifying a compound that modulates the stability of native-state protein (e.g., stabilizes the native-state of a protein). Such compounds can be used to treat a variety of diseases associated with misfolded or misassembled proteins.

The methods can be useful, for example, in identifying compounds that stabilize the native-state of a transthyretin. Such identified compounds can be useful in the treatment of subjects (e.g., humans (e.g., human patients)) having, or at risk of developing, a transthyretin amyloid disease (e.g., familial amyloid polyneuropathy, familial amyloid cardiomyopathy, senile systemic amyloidosis, cardiac amyloidosis following liver transplantation, peripheral nerve amyloidosis following liver transplantation, leptomeningeal amyloidosis, transthyretin mutant-associated carpal tunnel syndrome, vitreous deposition, or transthyretin mutant-associated skin amyloidosis). Examples of compounds useful as a positive control in the screening assay to identify compounds that stabilize the tetrameric forms of transthyretin are described below, and include, for example, genistein, thyroxin (T4), and derivatives of some non-steroidal anti-inflammatory drugs (NSAIDs) (e.g., diflunisal, diclofenac, flufenamic acid, and derivatives thereof) (Green et al. (2005) Proc. Natl. Acad. Sci. USA 102(41):14545-14550; Alves et al. (1997) Eur. J. Biochem. 249:662-668; and Almeida et al. (2005) Curr. Drug Targets CNS Neurol Disord. 4(5):587-596). Other compounds useful in the method include 2-(2,6-dichloro-phenyl)-benzoxazole-6-carboxylic acid and 2-(3,5-dichloro-phenyl)-benzoxazole-6-carboxylic acid (see the Examples below).

The methods can also be used to identify similar “pharmacological chaperones” (i.e., compounds that stabilize the native-state of proteins) for any other proteins described herein. Pharmacological chaperones identified for the cystic fibrosis transmembrane conductance regulator protein can be useful, for example, in the treatment of cystic fibrosis. Pharmacological chaperones identified for alpha-galactosidase A, beta-glucocerebrosidase, or alpha-glucosidase can be useful in the treatment of Fabry disease, Gaucher diseases, and Pompe disease, respectively. Examples of pharmacological chaperones can be found, e.g., in Sawkar et al. (2002) Proc. Natl. Acad. Sci. USA 99(24):15428-15433; Robert et al. (2005) J. Biol. Chem. 280(51):42198-42206; and Robben et al. (2006) Mol. Biol. Cell 17:379-386.

The above-described methods can be useful in identifying compounds that inhibit or reduce amyloid fibril formation by transthyretin. These compounds can be useful in the treatment of subjects (e.g., humans (e.g., human patients)) having, or at risk of developing familial amyloid polyneuropathy (FAP) and familial amyloid cardiomyopathy (FAC). Examples of such compounds are known to those of skill in the art and include, for example, 4′-iodo-4′deoxydoxorubicin (1-DOX) (Palha et al. (2000) Am. J. Pathol. 156:1919-1925).

Similarly, the methods can also be useful in identifying compounds that inhibit or reduce the formation of neurofibrillary tangles or filaments associated with disease-inducing amyloid forms of Alpha-Beta (AB) protein, alpha-synuclein, or Tau. Such identified compounds can be useful in the treatment of subjects (e.g., humans (e.g., human patients)) having, or at risk of developing Parkinson's disease, Alzheimer's disease or any other tauopathy (i.e., pathologies where disease-associated oligomer-forming polypeptide is a Tau polypeptide).

The screening methods can also serve to identify compounds useful in inhibiting or reducing the amyloid aggregation of prion proteins. These compounds can be useful in treating a subject having, or at risk of developing, a prion-related disease including, e.g., scrapie, transmissible mink encephalopathy, chronic wasting disease, bovine spongiform encephalopathy, feline spongiform encephalopathy, exotic ungulate encephalopathy prion, Kuru, Creutzfeldt-Jakob disease, Variant Creutzfeldt-Jakob disease, Gerstmann-Staussler-Scheinker syndrome, or fatal familial insomnia.

In instances where the native-state of a protein causes or contributes to a disease (e.g., overexpression of oncogenes like ras or myc in certain cancers), the immuno-turbidimetric screening assay method can be used to identify compounds that stimulate conversion of native-state protein to non-native-state protein.

The protein whose native-state causes or contributes to disease can be, for example, an oncogene. Such oncogenes include, but are not limited to, ras, myc, HER-2/neu (erB-2), hTERT, Bcl-2, src, raf, Bruton's agammaglobulinemia tyrosine kinase (BTK), platelet-derived growth factor receptor, vascular endothelial growth factor receptor, epidermal growth factor receptor, and aurora kinases. Compounds identified that destabilize the native-state of oncogenes (i.e., stimulate the conversion of native-state protein to non-native-state protein) can be useful, for example, in the treatment of cancers.

The protein whose native-state causes or contributes to disease can also be a viral protein. In this case, the method can be useful in identifying compounds that stimulate the dissociation (e.g., conversion of native-state protein to non-native-state protein) of oligomeric proteins (oligomer-forming polypeptides) of viral coats or capsids. Examples of such oligomeric proteins include, but are not limited to: the p24 capsid polypeptide of an HIV-1 virus; the VP5, VP19c, VP21, VP23, VP24, or VP26 capsid polypeptides of a Herpes Virus (e.g., HSV-1); the L1 capsid polypeptide of a Papillomaviruses (e.g., HPV-16 or HPV-18); or the VP1 capsid polypeptides of a Hepatitis viruses (e.g., Hepatitis-A virus). Compounds identified by the method can be useful in in vitro or in vivo methods of disrupting viral particle assembly or inhibiting infection of a cell by a virus. Where the oligomer-forming polypeptides are of the HIV-1 capsid (e.g., a p24 capsid protein of an HIV-1 virus), the compounds can be useful in treating a subject (e.g., a mammal, e.g., a human patient) infected, likely to become infected, or a risk of becoming infected with an HIV-1 virus.

In any of the methods described above, the protein can be contacted with the compound in vitro or in vivo. Where the protein is contacted with the compound in vivo, such methods can be used, for example, for screening of compounds in animal models of diseases (e.g., any of the transthyretin amyloid diseases described herein). Alternatively, such methods can be used for evaluating the efficacy of clinical candidate compounds administered to humans.

Screening assays can also be performed in any format that allows for rapid preparation, processing, and analysis of multiple reactions. This can be, for example, in multi-well assay plates (e.g., 96 wells or 386 wells). Stock solutions for various agents can be generated manually or robotically, and all subsequent pipetting, diluting, mixing, distribution, washing, incubating, sample readout, data collection and analysis can be done robotically using commercially available analysis software, robotics, and detection instrumentation (e.g., a spectrophotometer) capable of detecting the immuno-turbidimetric signal generated from the assay.

### Additional Methods for Detecting Native-State and Non-Native-State Proteins

Additional methods for detecting (e.g., measuring) the presence or amount of either non-native-state or native-state proteins (e.g., transthyretin) are described below in the Examples. The additional methods can be used, for example, in validation studies (e.g., as secondary or confirmatory tests run parallel with the immuno-turbidimetric assays). Alternatively, the additional methods can be used to detect the native-state or non-native-state proteins, for example, from the same sample or from parallel samples used for any of the immuno-turbidimetric assays described herein. Additional methods include, for example, high-performance liquid chromatography (HPLC), western blotting techniques, and circular dichroism. The presence or amount of native-state protein or non-native state protein can be determined by analyzing the rate of its physical passage through a stationary phase matrix (e.g., HPLC or thin-layer chromatography (TLC) methodology). For the HPLC method, samples can be resuspended in an appropriate solvent (or liquid phase) and actively or passively passaged over a stationary-phase, size-exclusion matrix, which can retard (i.e., increase the retention time of), for example, the native-state of a protein on the basis of physical properties (e.g., size, hydrophobicity or charge). Larger forms of protein (e.g., native-state oligomeric forms of an oligomer-forming polypeptide) can generally pass over the matrix uninhibited, whereas smaller (e.g., non-native-state monomeric forms) of a protein can get trapped in the small pores of the matrix, effectively slowing their flow-rate over the column. The presence, amount, or relative ratio of native-state protein can be determined by measuring the retention time between the passage of the native-state protein compared to non-native-state protein over the stationary phase matrix. For more details about HPLC methodology, see, for example, Nageswara-Rao et al. J. Pharm. Biomed. Anal. 2003 Oct. 15; 33(3):335-77). Alternatively, following the reaction step of the procedure, the mixture can be resuspended in Laemmli buffer and subjected to polyacrylamide gel electrophoresis (PAGE). The PAGE can also involve a detergent, like SDS, provided that the detergent does not disrupt the stability of native-state protein. PAGE-resolved protein complexes (e.g., native-state proteins when present in a sample), separated by size can then be transferred to a filter membrane (e.g., nitrocellulose) and subjected to western blot techniques using antibodies specific for the protein (as described above). The relative amount or ratio of native-state protein, optionally in the presence or absence of a test compound, can be detected (determined) by comparison to the relative amount of the non-native-state protein. In some cases, adjustments are made to correct for reactivity differences between different forms of the protein. Examples of western-blotting variations of the methods are described in the Examples below.

Circular dichroism (CD) spectroscopy measures or detects differences in the absorption of left-handed polarized light versus right handed polarized light which arise due to structural asymmetry. The absence of regular structure results in zero CD intensity, while an ordered structure results in a spectrum which can contain both positive and negative signals. Changes in secondary structure, monitored in the far-UV CD region, can be determined with as little as 50 μg of protein, at concentrations of 0.2 mg/ml. Detection of native-state and non-native-state protein generally works best if the individual forms (i.e., native-state or non-native-state) of the protein have CD spectra which are quite different from each other, such that changes at specific wavelengths can be monitored to follow changes in the corresponding protein. In such cases, it is also possible to determine an increase in stability of native-state protein following, for example, oligomerization or denaturation of the protein (see, for example, McCann et al. (2006) J. Org. Chem. 71(16):74-98; Stephens et al. (2006) J. Nat. Prod. 69(7):1055-1064; and Oakley et al. (2006) Chirality 18(5):340-347).

### Compounds

Compounds that can be screened by the methods described herein include various chemical classes and are typically small organic molecules having a molecular weight in the range of 50 to 2,500 daltons. These compounds can comprise functional groups necessary for structural interaction with proteins (e.g., hydrogen bonding), and typically include at least an amine, carbonyl, hydroxyl, or carboxyl group, and preferably at least two of the functional chemical groups. These compounds often comprise cyclical carbon or heterocyclic structures and/or aromatic or polyaromatic structures (e.g., purine core) substituted with one or more of the above functional groups.

The compounds can also include biomolecules including, but not limited to, peptides, polypeptides, peptidomimetics (e.g., peptoids), amino acids, amino acid analogs, saccharides, fatty acids, steroids, purines, pyrimidines, derivatives or structural analogues thereof, polynucleotides, and polynucleotide analogs.

Other examples of small molecule compounds are nucleic acid aptamers which are relatively short nucleic acid (DNA, RNA or a combination of both) sequences that bind with high avidity to a variety of proteins and inhibit the binding to such proteins of ligands, receptors, and other molecules. Aptamers are generally about 25-40 nucleotides in length and have molecular weights in the range of about 18-25 kDa. Aptamers with high specificity and affinity for targets can be obtained by an in vitro evolutionary process termed SELEX (systemic evolution of ligands by exponential enrichment) [see, for example, Zhang et al. (2004) Arch. Immunol. Ther. Exp. 52:307-315, the disclosure of which is incorporated herein by reference in its entirety]. For methods of enhancing the stability (by using nucleotide analogs, for example) and enhancing in vivo bioavailability (e.g., in vivo persistence in a subject's circulatory system) of nucleic acid aptamers see Zhang et al. (2004) and Brody et al. [(2000) Reviews in Molecular Biotechnology 74:5-13, the disclosure of which is incorporated herein by reference in its entirety].

Compounds can be identified from a number of potential sources, including: chemical libraries, natural product libraries, and combinatorial libraries comprised of random peptides, oligonucleotides, or organic molecules. Chemical libraries consist of a diversity of chemical structures, some of which are analogs of known compounds or analogs or compounds that have been identified as “hits” or “leads” in other drug discovery screens, while others are derived from natural products, and still others arise from non-directed synthetic organic chemistry. Natural product libraries are collections of microorganisms, animals, plants, or marine organisms which are used to create mixtures for screening by: (1) fermentation and extraction of broths from soil, plant or marine microorganisms, or (2) extraction of plants or marine organisms. Natural product libraries include polypeptides, non-ribosomal peptides, and variants (non-naturally occurring) thereof. For a review, see Science 282:63-68 (1998). Combinatorial libraries are composed or large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of test compounds through the use of the various libraries herein permits subsequent modification of the test compound “hit” or “lead” to optimize the capacity of the “hit” or “lead” to, for example, stabilize the native-state of a protein.

In addition, the inhibitory compounds can be antibodies, or antigen-binding antibody fragments, specific for a protein (e.g., an antibody or antigen-binding fragment specific for transthyretin). The antibody can be a purified or a recombinant antibody, or antibody fragments and chimeric antibodies and humanized antibodies made from non-human (e.g., mouse, rat, gerbil, or hamster) antibodies.

The compounds identified above can be synthesized by any chemical or biological method. The compounds identified above can also be pure, or may be in a heterologous composition (e.g., a pharmaceutical composition), and can be prepared in an assay-, physiologic-, or pharmaceutically-acceptable diluent or carrier. These compositions can also contain additional compounds or constituents which do not stabilize native-state protein, but are useful in the application of various methods described herein (e.g., a composition may contain one or more solvents, diluents, or buffers).

Exemplary compounds useful in any of the methods described herein as, for example, positive controls, are compounds known to stabilize native-state protein. Compounds that are known to stabilize tetrameric transthyretin complexes include, for example, genistein and thyroxin (T4) (Green et al. (2005) Proc. Natl. Acad. Sci. USA 102(41):14545-14550; and Alves et al. (1997) Eur. J. Biochem. 249:662-668). Other compounds useful as positive controls in methods of identifying compounds that stabilize a transthyretin tetramer include 2-(2,6-dichloro-phenyl)-benzoxazole-6-carboxylic acid and 2-(3,5-dichloro-phenyl)-benzoxazole-6-carboxylic acid, or any other transthyretin-stabilizing compound disclosed in U.S. Publication No. US 2004/0152140 A1, which is incorporated herein by reference in its entirety.

The following examples are intended to illustrate, not limit, the invention.

### EXAMPLES

### Example 1

### Determining Transthyretin Tetramer Stability Using Immuno-Turbidimetric Assay 1

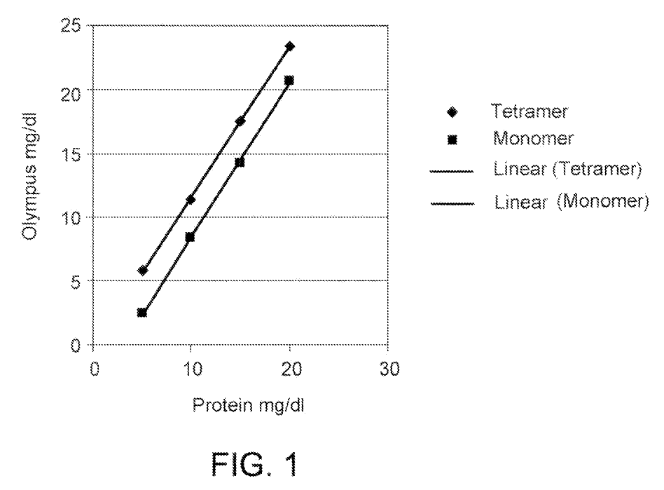

To assay for the ability of an anti-transthyretin antibody to detect different forms of transthyretin (monomeric or tetrameric forms of transthyretin), samples containing various concentrations [5-20 mg/dL] of either (a) tetrameric transthyretin, or (b) monomeric transthyretin (a transthyretin polypeptide containing two point mutations in the dimerization domain which prevents oligomer assembly; see Jiang et al. (2001) Biochemistry 40(38): 11442-52) were incubated with a commercially available transthyretin-specific antibody, OSR6175 reagent (Olympus Life and Material Science Europa GmbH, Ireland)), and the turbidity, as a measure of the difference between incident and transmitted light, was calculated per manufacturer's instructions. OSR6175 accurately measured the known concentration of tetrameric transthyretin in each sample (FIG. 1). OSR6175 also detected the known concentration of monomeric transthyretin in each of the samples, although the correlation between known input amount and calculated amount was not 1:1 (FIG. 1).

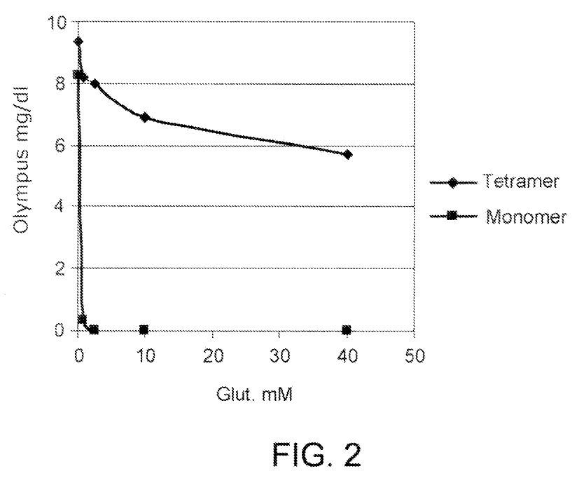

Three day treatment of transthyretin containing samples with urea resulted in nearly complete loss of detection of transthyretin using the immuno-turbidimetric assay. In view of this finding, tetrameric transthyretin was preserved in a sample by treatment with the protein cross-linking agent glutaraldehyde. To determine the ability of OSR6175 to detect different forms of transthyretin in the presence of glutaraldehyde, samples containing 10 mg/dL of either tetrameric or monomeric transthyretin (see above) were incubated with glutaraldehyde at various final concentrations from 0 to 40 mM. The mixtures were incubated for 4 minutes at room temperature. The cross-linking reaction was stopped by the addition of 54 mL of 1.85 M freshly prepared sodium borohydride in 0.1 N sodium hydroxide, followed by a 5 minute incubation at room temperature. Tetrameric and monomeric transthyretin were readily detected in the samples at 0 mM glutaraldehyde (FIG. 2). However, upon treatment with glutaraldehyde, the detection of monomeric was severely attenuated, whereas tetrameric transthyretin was detectable at all concentrations of glutaraldehyde tested (FIG. 2). These results indicated that the OS6175 reagent could detect tetrameric transthyretin and distinguish tetrameric versus monomeric transthyretin, in a sample in the presence of at least up to 40 mM glutaraldehyde.

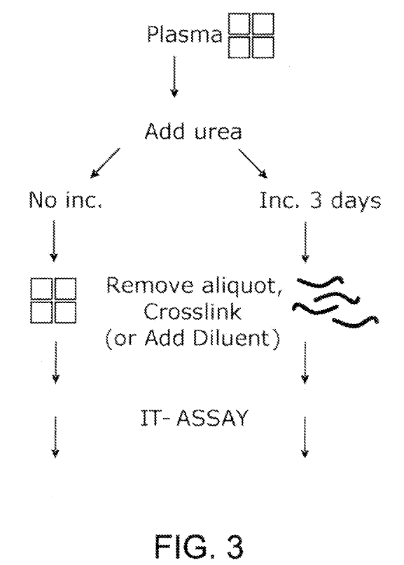

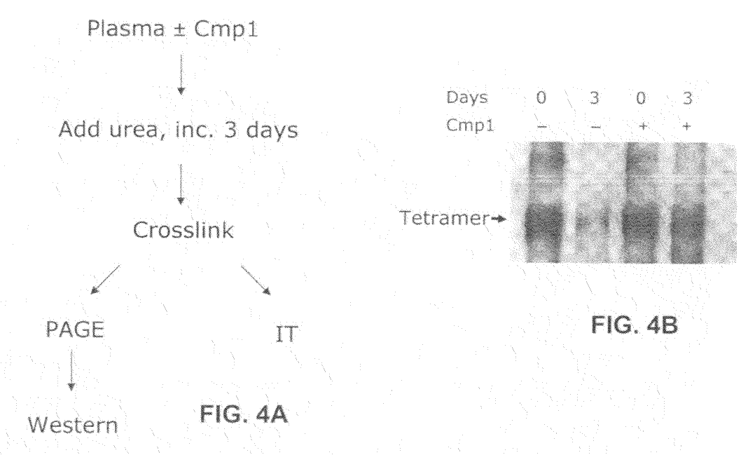

Next, the immuno-turbidity assay was evaluated in human plasma samples containing transthyretin and in the presence and absence of a test compound 2-(2,6-dichloro-phenyl)-benzoxazole-6-carboxylic acid (a transthyretin stabilizing compound, see Published U.S. Application No. 20040152140). A flow chart of the experimental method is provided in FIG. 3. A 1 mL plasma sample was incubated with 1 μL of 2-(2,6-Dichloro-phenyl)-benzoxazole-6-carboxylic acid (solubilized in dimethyl sulfoxide (DMSO), or a control solution containing only DMSO, for 15 minutes at room temperature. The final test concentration of 2-(2,6-dichloro-phenyl)-benzoxazole-6-carboxylic acid (Cmp. 1) was 7.2 μM. Experimental (with compound) or control (only DMSO) samples were then each combined with 1.5 mL of 8 M urea to a final urea concentration of 4.8 M. A 1 mL aliquot was removed immediately (no incubation (no-inc)) for cross-linking and the remaining 1.5 mL mixture was incubated for 3 days at room temperature (3 day). Immediately for the no-inc 1 mL aliquot or following the 3 day incubation for the 3 day samples, 1 mL of the samples was cross-linked by adding 16 L of 25% (2.5 M) glutaraldehyde in water to a final concentration of 40 mM. The mixtures were incubated for 4 minutes at room temperature. The cross-linking reaction was stopped by the addition of 54 μL of 1.85 M freshly prepared sodium borohydride in 0.1 N sodium hydroxide, followed by a 5 minute incubation at room temperature. For analysis, the OSR6175 reagent was added to sample per manufacturer's instructions, and the turbidity, as a measure of the difference between incident and transmitted light, was calculated per manufacturer instruction. Three-day treatment of the samples with urea caused almost a complete loss of tetrameric transthyretin (Table 1). In contrast, pretreatment of the samples with 2-(2,6-dichloro-phenyl)-benzoxazole-6-carboxylic acid nearly completely stabilized tetrameric transthyretin. These results were compared to an alternative, western blotting method. The method was performed as described above, however, following the cross-linking step, samples were solubilized in Laemmli buffer and subjected to sodium dodecyl sulfate-polyacrylamide gel electrophoresis (SDS-PAGE) (FIG. 4A). Size-resolved proteins were transferred from the gel to nitrocellulose and immunoblotted using antibodies specific for transthyretin. As shown in FIG. 4B, and in agreement with the OSR6175 immuno-turbidity assay, 3 day treatment of the sample with urea nearly completely abolished tetrameric transthyretin, whereas tetrameric transthyretin was almost completely stabilized in the presence of 2-(2,6-dichloro-phenyl)-benzoxazole-6-carboxylic acid.

TABLE 1
Effect of Cmp. 1 on Transthyretin Tetramer Stability (Immuno-
turbidimetric Assay No. 1)

| | | | Normalized |
|---|---|---|---|
| Glut. mM | Day | Comp. 1 | (mg/dL) |
| 40 | 0 | − | 22.7 |
| 40 | 3 | − | 1.07 |
| 40 | 0 | + | 23.5 |
| 40 | 3 | + | 21.9 |

Glut. is glutaraldehyde, expressed in concentration units of millimolar (mM).
Cmp. 1 is Compound 1, or 2-(2,6-dichloro-phenyl)-benzoxazole-6-carboxylic acid, and was used at a concentration of 7.2 μM.
“+” indicates the presence of and
“−” indicates the absence of the Compound in the Sample.
Normalized refers to a mathematical correction of the Olympus (IT) reagent determination of protein concentration, adjusting for the dilution step of the assay.

### Example 2

### Determining Transthyretin Tetramer Stability Using Immuno-Turbidimetric Assay 2

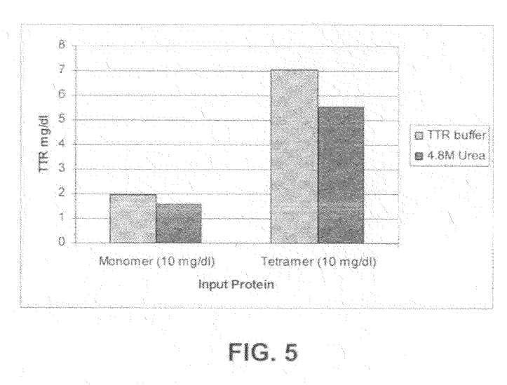

The first immuno-turbidity assay format (i.e., the Immuno-turbidimetric Assay 1 above) contains a cross-linking step that eliminates monomer recognition (see FIG. 2). An alternate form of the assay was developed using antibody reagents that distinguish monomeric and tetrameric forms of transthyretin. One example of such a reagent is the anti-transthyretin antibody reagent of the Beckman Prealbumin kit (Prealbumin PAB #475106, Beckman Coulter, Fullerton, Calif.). The selectivity of the Beckman Prealbumin kit antibody reagent was demonstrated by assaying solutions of purified recombinant tetramer or monomer as described by the manufacturer. Both preparations contained 10 mg/dL transthyretin as measured by A280. Using the non-cross-linking immuno-turbidimetric assay format, tetrameric transthyretin was detected preferentially to monomeric transthyretin; this selectivity was observed in the presence or absence of 4.8 M urea (FIG. 5). The selectivity of transthyretin immuno-turbidimetric reagents of the ADVIA prealbumin kit (Bayer Corporation, Diagnostics Division, Tarrytown, N.Y.) were also tested. Solutions containing 20 mg/dL of either purified recombinant transthyretin tetramer or monomer were assayed using the protocol provided with the Bayer kit. Concentration values, determined from this kit, were 22.3 and 0.62 mg/dL, for the tetramer and monomer respectively. Thus, the Bayer reagents demonstrate strong selectively for the transthyretin tetramer, displaying a greater than 20-fold differential binding affinity for the transthyretin tetramer over monomer.

In this second assay, the cross-linking step is replaced with a step of terminating denaturation (caused by the urea treatment) by dilution of the sample. Briefly, a 1 ml sample of human plasma was combined with either 1 μl of a test compound (Cmp. 1: 2-(2,6-dichloro-phenyl)-benzoxazole-6-carboxylic acid or Cmp. 2: 2-(3,5-dichloro-phenyl)-benzoxazole-6-carboxylic acid; see Published U.S. Application No. 20040152140) solubilized in DMSO or a control solution of DMSO, and the samples were incubated for 15 minutes at room temperature prior to addition of urea. The final concentration of the test compounds was 7.2 μM in DMSO. A solution of urea was added such that the final concentration of urea in the sample was 4.8 M. A 1 mL aliquot was removed immediately (no incubation (no-inc)) for dilution (see below) and the remaining 1.5 mL mixture was incubated for 3 days at room temperature (3 day) (FIG. 3). Immediately (for the no-inc) or following the three day incubation (for the 3 day samples), the samples were diluted 1:5 (four volumes diluent were added per one volume equivalent of sample). Potassium chloride (KCl) and sodium phosphate (NaPO4) were also added to the samples at final concentrations of 50 mM and 25 mM, respectively.

As shown in Table 2, and consistent with the previous immuno-tubidimetric assay format, incubation of the plasma samples with urea for 3 days results in near complete loss of tetrameric transthyretin (7.3 mg/dL at day 0 to ˜0.8 mg/dL at day 3). However, in the presence of 7.2 μM Cmp. 1, the transthyretin tetramer was almost completely stabilized. Similarly, Cmp. 2 significantly stabilized the transthyretin tetramer in two duplicate experiments (Table 2).

TABLE 2
Effect of Test Compounds on Transthyretin Stability (Immuno-
turbidimetric Assay No. 2)

| | | | Olympus | Beckman |
|---|---|---|---|---|
| | | | Normalized | Normalized |
| Comp. | Comp. μM | Day | (mg/dL) | (mg/dL) |
| 0 | 0 | 0 | 19.45 | 10.53 |
| 0 | 0 | 3 | 2.00 | 4.13 |
| Cmp. 1 | 7.2 | 0 | 22.12 | 10.53 |
| Cmp. 1 | 7.2 | 3 | 23.45 | 12.26 |
| Plasma | 0 | 0 | 24.7 | 27.3 |
| Cmp. 2 | 7.2 | 0 | 22.12 | |
| Cmp. 2 | 7.2 | 3 | 13.59 | |
| Cmp. 2 | 7.2 | 0 | 23.72 | |
| Cmp. 2 | 7.2 | 3 | 13.59 | |

Cmp. 1 is Compound 1, or 2-(2,6-dichloro-phenyl)-benzoxazole-6-carboxylic acid, and was used at a concentration of 7.2 μM.
Cmp. 2 is Compound 2, or 2-(3,5-dichloro-phenyl)-benzoxazole-6-carboxylic acid, and was used at a concentration of 7.2 μM.
Normalized refers to a mathematical correction of the determination of protein concentration, adjusting for the dilution step of the assay

### Example 3

### Dose-Dependent Stabilization of Transthyretin

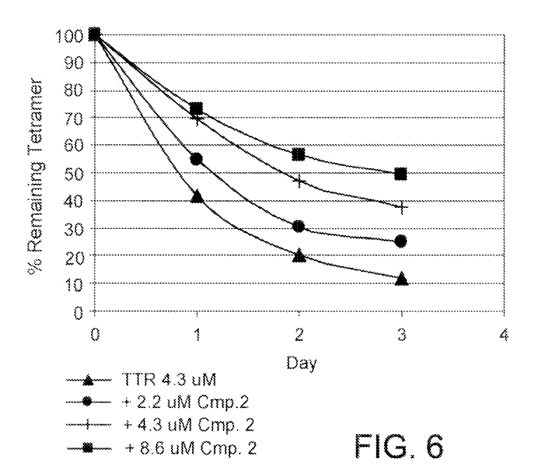

To test the effect of different dosages of 2-(3,5-dichloro-phenyl)-benzoxazole-6-carboxylic acid (Cmp. 2) on transthyretin stabilization, plasma samples (a pool from 20 individual donors) containing 4.3 M transthyretin were treated with 2.2 μM, 4.3 μm, or 8.6 μm Cmp. 2, or DMSO as a negative control, for 15 minutes at room temperature. Following the incubation, the urea denaturant was added to the samples resulting in a final urea concentration of 4.8 M (see FIG. 6). As above, aliquots were removed immediately (day 0) and after incubation at room temperature for 1, 2, and 3 days. Cross-linking (glutaraldehyde) and detection of transthyretin using the Olympus OSR6175 reagent were performed as described under Example 1. For each concentration of Cmp. 2, the transthyretin level at days 1, 2, and 3 was divided by the initial concentration at day 0 to yield the percent of tetramer remaining (FIG. 6). These experiments indicated that the higher concentrations of Cmp. 2 (8.6 μM Cmp 2, squares of FIG. 6) were more effective than lower concentrations of Cmp. 2 (2.2 μM Cmp. 2, circles of FIG. 6) at stabilizing tetrameric transthyretin (FIG. 6).

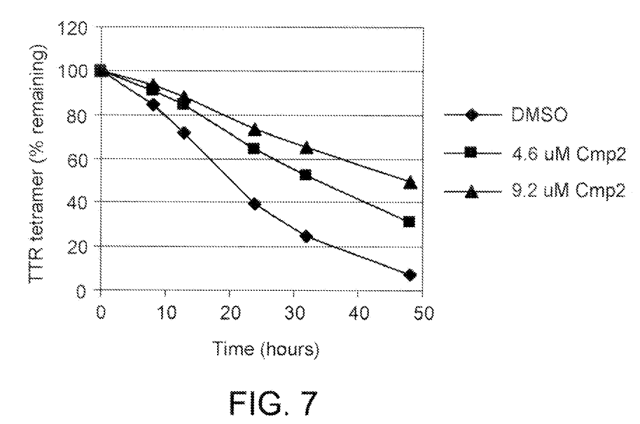

Dose-dependent stabilization of transthyretin by Cmp. 2 was also monitored using immuno-turbidimetric assay 2 (Example 2). Plasma samples (a pool from 16 individual donors) containing 4.6 μM transthyretin were treated with 4.6 μm, or 9.2 μm Cmp. 2, or DMSO as a negative control, for 15 minutes at room temperature. Following the incubation, urea was added to the samples resulting in a final urea concentration of 3.2 M (see FIG. 7). Aliquots were removed immediately (day 0) and after incubation at room temperature for 8, 13, 24, 32, and 48 hours. Immediate detection of transthyretin using the Bayer ADVIA prealbumin kit was performed as described by the manufacturer. For each concentration of Cmp. 2, the assay-determined transthyretin concentration at 8, 13, 24, 32, and 48 hours was divided by the assay-determined initial concentration at day 0 to yield the percent of tetramer remaining (FIG. 7). Higher concentrations of Cmp. 2 were more effective than lower concentrations of Cmp. 2 at stabilizing tetrameric transthyretin (FIG. 7).

### Example 4

### Efficacy of Compound 2 Against Different Sample Concentrations of Transthyretin

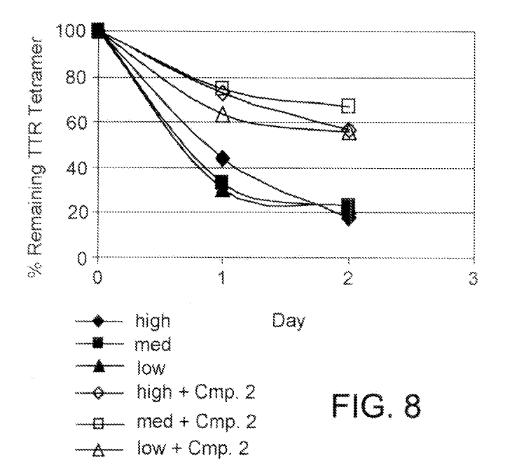

Since the concentration of transthyretin fluctuates from day to day in an individual subject and can vary between different subjects (e.g., different patients), the efficacy of Cmp. 2 to stabilize tetrameric transthyretin was tested for different concentrations of transthyretin. Three plasma samples containing 17.7 mg/dL transthyretin (a pool of two individuals; designated as “low”), 25.4 mg/dL transthyretin (a pool of 16 individuals; designated as “medium”), or 30.1 mg/dL transthyretin (a pool of two individuals; designated as “high”) were pre-treated with 7.2 μM Cmp. 2 or DMSO for 15 minutes at room temperature. Samples were taken either immediately (day 0) or at 1 day intervals of 1, 2, or 3 days. Samples were then subjected to denaturation and cross-linking, and stabilized tetrameric transthyretin was measured as described above in Examples 1 and 3. In the presence and absence of Cmp. 2, and at day 1, 2, and 3, the tetrameric transthyretin concentration at days 1, 2, and 3 was divided by the initial concentration at day 0 to yield the percent of tetramer remaining (FIG. 8). These experiments showed not only that 7.2 mM Cmp. 2 was equivalently effective on different concentrations of transthyretin, but also that the kinetics of tetrameric transthyretin stabilization in the presence of 7.2 μM Cmp. 2 were equivalent (FIG. 8).

### Example 5

### Efficacy of Compound 2 Against Samples from Patients with Mutant TTR (V30M)

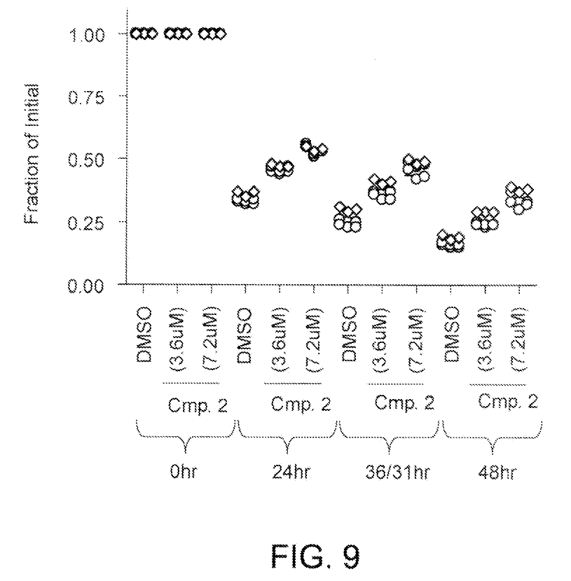

The ability of Cmp. 2 to stabilize tetrameric transthyretin in plasma samples from familial amyloid polyneuropathy patients harboring the V30M transthyretin mutation was tested. Aliquots of pooled plasma samples (from nine individuals) containing a final concentration of 22 mg/dL of transthyretin were first treated with 3.6 M or 7.2 M of Cmp. 2 or with DMSO for 15 minutes at room temperature. The samples were then subjected to denaturation for 24, 31 or 36, or 48 hours and then cross-linked (as above). Stabilized tetrameric transthyretin was measured as described above in Examples 1 and 3. In the presence and absence of Cmp. 2, the tetrameric transthyretin concentration at 24, 31 or 36, or 48 hours was divided by the initial concentration present in the untreated samples to yield the percent of tetramer remaining (FIG. 9). These experiments demonstrated that tetrameric transthyretin in plasma samples from patients harboring a V30M transthyretin mutation is stabilized in a dose-dependent manner by Cmp. 2.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of detecting the presence or amount of a stabilized native-state protein in a sample, the method comprising:
contacting a sample comprising a protein with a denaturant for a time sufficient to induce conversion of unstabilized native-state protein to a non-native-state of the protein, resulting in mixture (a);
contacting mixture (a) with a cross-linking agent for a time sufficient to cross-link the protein, resulting in mixture (b);
contacting mixture (b) with an antibody that specifically binds to the protein to form mixture (c), wherein (i) the antibody preferentially binds to cross-linked native-state protein as compared to cross-linked non-native-state protein and binding of the antibody to cross-linked native-state protein results in the formation of insoluble aggregates of the antibody and cross-linked native-state protein, or (ii) the antibody preferentially binds to cross-linked non-native-state protein as compared to cross-linked native-state protein and binding of the antibody to cross-linked non-native-state protein results in the formation of insoluble aggregates of the antibody and cross-linked non-native-state protein; and
detecting the presence or amount of insoluble aggregates in mixture (c), wherein detection of insoluble aggregates indicates directly or indirectly the presence or amount of stabilized native-state protein in the sample.

2. A method of detecting the presence or amount of a stabilized native-state protein in a sample, the method comprising:
contacting a sample comprising a protein with a denaturant for a time sufficient to induce conversion of unstabilized native-state protein to a non-native-state of the protein, resulting in mixture (a);
contacting mixture (a) with an antibody that specifically binds to the protein to form mixture (b), wherein (i) the antibody preferentially binds to native-state protein as compared to non-native-state protein and binding of the antibody to native-state protein results in the formation of insoluble aggregates of the antibody and native-state protein, or (ii) the antibody preferentially binds non-native state protein and binding of the antibody to non-native-state protein results in the formation of insoluble aggregates of the antibody and non-native-state protein; and
detecting the presence or amount of insoluble aggregates in mixture (b), wherein detection of insoluble aggregates indicates directly or indirectly the presence or amount of stabilized native-state protein in the sample.

3. The method of claim 1, wherein detection of insoluble aggregates is a direct indication of the presence or amount of stabilized native-state protein in the sample.

4. The method of claim 1, wherein detection of insoluble aggregates is an indirect indication of the presence or amount of stabilized native-state protein in the sample, the presence or amount of stabilized native-state protein in the sample being inversely proportional to the insoluble aggregates detected.

5. The method of claim 2, further comprising contacting mixture (a) with a diluent prior to contacting mixture (a) with the antibody.

6. The method of claim 1, wherein the sample further comprises a candidate compound.

7. A method of identifying a compound that stabilizes the native-state of a protein, the method comprising:
contacting a sample comprising a protein and a candidate compound with a denaturant for a time sufficient to induce conversion of unstabilized native-state protein to a non-native-state of the protein, resulting in mixture (a);
contacting mixture (a) with a cross-linking agent for a time sufficient to cross-link the protein, resulting in mixture (b);
contacting mixture (b) with an antibody that specifically binds to the protein to form mixture (c), wherein (i) the antibody preferentially binds to cross-linked native-state protein as compared to cross-linked non-native-state protein and binding of the antibody to cross-linked native-state protein results in the formation of insoluble aggregates of the antibody and cross-linked native-state protein, or (ii) the antibody preferentially binds to cross-linked non-native-state protein as compared to cross-linked native-state protein and binding of the antibody to cross-linked non-native-state protein results in the formation of insoluble aggregates of the antibody and cross-linked non-native-state protein; and
detecting the presence or amount of insoluble aggregates in mixture (c), wherein (i) an increase in the formation of insoluble aggregates of the antibody and cross-linked native-state protein in the presence of the candidate compound as compared to in the absence of the candidate compound indicates that the candidate compound stabilizes the native-state of the protein, or (ii) a decrease in the formation of insoluble aggregates of the antibody and cross-linked non-native state protein in the presence of the candidate compound as compared to in the absence of the candidate compound indicates that the candidate compound stabilizes the native-state of the protein.

8. A method of identifying a compound that stabilizes the native-state of a protein, the method comprising:
contacting a sample comprising a protein and a candidate compound with a denaturant for a time sufficient to induce conversion of unstabilized native-state protein to a non-native-state of the protein, resulting in mixture (a);
contacting mixture (a) with an antibody that specifically binds to the protein to form mixture (b), wherein (i) the antibody preferentially binds to native-state protein as compared to non-native-state protein and binding of the antibody to native-state protein results in the formation of insoluble aggregates of the antibody and native-state protein, or (ii) the antibody preferentially binds non-native state protein and binding of the antibody to non-native-state protein results in the formation of insoluble aggregates of the antibody and non-native-state protein; and
detecting the presence or amount of insoluble aggregates in mixture (b), wherein (i) an increase in the formation of insoluble aggregates of the antibody and native-state protein in the presence of the candidate compound as compared to in the absence of the candidate compound indicates that the candidate compound stabilizes the native state of the protein, or (ii) a decrease in the formation of insoluble aggregates of the antibody and non-native-state protein in the presence of the candidate compound as compared to in the absence of the candidate compound indicates that the candidate compound stabilizes the native state of the protein.

9. The method of claim 8, further comprising contacting mixture (a) with a diluent prior to contacting mixture (a) with the antibody.

10. The method of claim 1, further comprising measuring the amount of insoluble aggregates.

11. The method of claim 7, wherein the protein is contacted with the candidate compound in vitro.

12. The method of claim 7, wherein the protein is contacted with the candidate compound in vivo.

13. The method of claim 1, wherein the protein is transthyretin.

14. The method of claim 13, wherein transthyretin is wild-type, mature transthyretin.

15. The method of claim 13, wherein transthyretin is an amyloidogenic form of transthyretin.

16. The method of claim 15, wherein the amyloidogenic form of transthyretin is the V30M mutant form of transthyretin.

17. The method of claim 15, wherein amyloidogenic form of transthyretin is the V122I mutant form of transthyretin, the Y78F mutant form of transthyretin, the L55P mutant form of transthyretin, the L55Q mutant form of transthyretin, the A25T mutant form of transthyretin, the C10R mutant form of transthyretin, the L12P mutant form of transthyretin, the D18E mutant form of transthyretin, the D18G mutant form of transthyretin, the V20I mutant form of transthyretin, the S23N mutant form of transthyretin, the P24S mutant form of transthyretin, the V28M mutant form of transthyretin, the V30A mutant form of transthyretin, the V30L mutant form of transthyretin, the V30L mutant form of transthyretin, the V30G mutant form of transthyretin, the F33I mutant form of transthyretin, the F33L mutant form of transthyretin, the F33V mutant form of transthyretin, the R34T mutant form of transthyretin, the L35N mutant form of transthyretin, the A36P mutant form of transthyretin, the D38A mutant form of transthyretin, the E42G mutant form of transthyretin, the E42D mutant form of transthyretin, the F44S mutant form of transthyretin, the A45D mutant form of transthyretin, the A45S mutant form of transthyretin, the A45T mutant form of transthyretin, the G47R mutant form of transthyretin, the G47A mutant form of transthyretin, the G47V mutant form of transthyretin, the G47E mutant form of transthyretin, the T49A mutant form of transthyretin, the T49I mutant form of transthyretin, the S50R mutant form of transthyretin, the S50I mutant form of transthyretin, the E51G mutant form of transthyretin, the S52P mutant form of transthyretin, the G53E mutant form of transthyretin, the E54G mutant form of transthyretin, the E54K mutant form of transthyretin, the L55R mutant form of transthyretin, the H56R mutant form of transthyretin, the L58H mutant form of transthyretin, the L58R mutant form of transthyretin, the T59K mutant form of transthyretin, the T60A mutant form of transthyretin, the E61K mutant form of transthyretin, the F64L mutant form of transthyretin, the F64S mutant form of transthyretin, the I68L mutant form of transthyretin, the Y69H mutant form of transthyretin, the K70N mutant form of transthyretin, the V71A mutant form of transthyretin, the I73V mutant form of transthyretin, the S77F mutant form of transthyretin, the S77Y mutant form of transthyretin, the I84S mutant form of transthyretin, the I84N mutant form of transthyretin, the I184T mutant form of transthyretin, the E89Q mutant form of transthyretin, the E89K mutant form of transthyretin, the A91S mutant form of transthyretin, the A97G mutant form of transthyretin, the A97S mutant form of transthyretin, the I107V form of transthyretin, the I107M mutant form of transthyretin, the A109S mutant form of transthyretin, the L111M mutant form of transthyretin, S112I mutant form of transthyretin, the Y114C mutant form of transthyretin, the Y114H mutant form of transthyretin, the Y116S mutant form of transthyretin, the A120S mutant form of transthyretin, the Val122 deletion mutant form of transthyretin, or the V122A mutant form of transthyretin.

18. The method of claim 13, wherein the native-state of transthyretin is a tetramer.

19. The method of claim 1, wherein the protein is an amyloidogenic protein.

20. The method of claim 19, wherein the amyloidogenic protein is selected from the group consisting of beta-amyloid, alpha-synuclein, tau, an immunoglobulin light chain, serum amyloid A protein, serum amyloid P protein, apoA-I, beta2-microglobulin, gelsolin, a lysozyme, insulin, fibrinogen, a prion protein, superoxide dismutase, calcitonin, cystatin C, and atrial natriuretic peptide.

21. The method of claim 1, wherein the protein is a trafficking-defective protein.

22. The method of claim 21, wherein the trafficking-defective protein is the AF508 mutant form of cystic fibrosis transmembrane conductance regulator protein.

23. The method of claim 21, wherein the trafficking-defective protein is alpha-galactosidase A, beta-glucocerebrosidase, or alpha-glucosidase.

24. The method of claim 21, wherein the trafficking-defective protein is gonadotropin-releasing hormone receptor, water-channel aquaporin-2, alpha1-antitrypsin, alpha1-antitrypsin variant, alpha-subunit of hNav1.5, nephrin, multi-drug resistance protein 2, the PHEX gene product, pendrin, sulfonylurea receptor 1, AE1, ceruloplasmin, palmitoyl protein thioesterase 1, cartilage oligomeric matrix protein, the ELOVL4 gene product, aspartyl-glucosaminidase, connexin 32, rhodopsin, cystic fibrosis transmembrane conductance regulator protein, HFE, tyrosinase, protein C, complement C1 inhibitor, alpha-D-galactosidase, beta-hexosaminidase, sucrase-isomaltase, UDP-glucuronosyl-transferase, insulin receptor, growth hormone receptor, myeloperoxidase, preproparathyroid hormone, Human Ether-a-go-go-Related gene, tyroxine binding globulin, lipoprotein lipase, low-density lipoprotein receptor, microsomal triglyceride transfer protein, apolipoprotein a, thyroglobulin, type I pro-collagen, fibrinogen, alpha 1-antichymotrypsin, phosphoinositide-dependent kinase 1, phosphoinositide-dependent kinase 2, vasopressin precursor protein prepro-vasopressin neurophysin II, peripheral myelin protein 22, proteolipid protein, presenilin, von Willebrand factor, ERGIC-53/p58, or sedlin.

25. The method of claim 1, wherein the protein is an oligomer-forming polypeptide.

26. The method of claim 25, wherein the oligomer-forming polypeptide is selected from the group consisting of Alpha-Beta protein, superoxide dismutase, Abri/ADan, glial fibrillary acidic protein, ATP7B, hemoglobin, amyloid A, beta-2-microglobulin, custatin C, lysozyme, fibrinogen, AH and AL immunoglobulin proteins, ApoAI, ApoAII, gelsolin, lactoferrin, lactohedrin, survivin, EGF-R, Erb-B2, and IL-12.

27. The method of claim 1, wherein the sample is obtained from a mammal.

28. The method of claim 27, wherein the mammal is a human.

29. The method of claim 28, wherein the human has, or is at risk of developing, a transthyretin amyloid disease.

30. The method of claim 29, wherein the transthyretin amyloid disease is familial amyloid polyneuropathy, familial amyloid cardiomyopathy, senile systemic amyloidosis, cardiac amyloidosis following liver transplantation, peripheral nerve amyloidosis following liver transplantation, leptomeningeal amyloidosis, transthyretin mutant-associated carpal tunnel syndrome, vitreous deposition, or transthyretin mutant-associated skin amyloidosis.

31. The method of claim 28, wherein the human has, or is at risk of developing, a disorder selected from the group consisting of Alzheimer's disease, Dutch cerebrovascular amyloidosis, Flemish cerebrovascular amyloidosis, Italian cerebrovascular amyloidosis, progressive supranuclear palsy, progressive subcortical gliosis, Pick's disease, dementia pugilisticas, Parkinson's Disease, kuru, Creutzfeldt-Jakob disease, Alexander Disease, Wilson Disease, Lou Gehrig's Disease, sickle cell anemia, cystic fibrosis, diabetes, and Huntington's disease.

32. The method of claim 28, wherein the human has, or is at risk of developing, a lysosomal storage disease.

33. The method of claim 32, wherein the lysosomal storage disease is Fabry disease, Gaucher disease, Pompe disease, Hurler-Scheie syndrome, MPS type I, GM1gangliosidosis, galactosialidosis, Morquio syndrome B, MPS type IVB, Sandhoff disease, Tay-Sachs disease, beta-mannosidosis, alpha-L-fucosidosis, Maroteaux-Lamy syndrome, MPS type VI, metachromatic leukodystrophy, Schindler disease, aspartylglycosaminuria, Hunter syndrome, MPS type II, Sanfilippo syndrome A, MPS type IIIA, Sanfilippo syndrome B, MPS type IIIB, Sanfillipo syndrome C, MPS type IIIC, Sanfilippo syndrome D, MPS type IID, Morquio syndrome A, MPS type IVA, Sly syndrome, MPS type VII, hyaluronidase deficiency, MPS type IX, multiple sulfatase deficiency, alpha-mannosidosis, sialidosis, X-linked ictiosis and multiple sulfatase deficiency, mucolipidodisis II, mucolipidosis III, Wolman disease, Farber disease, Niemann-Pick disease A, Niemann-Pick disease B, glycogenosis type II, neuronal ceroid lipofucsinosis infantile type, neuronal ceroid lipofucsinosis late infantile type, neuronal ceroid lipofucsinosis juvenile type, Krabbe disease, lysosomal acid phosphatase deficiency, pycnodysostosis, cystinosis, sialic acid storage disease, cobalamin deficiency type F, Niemann-Pick disease type C, galactosialidosis, metachromatic leukodystrophy variant, Gaucher disease variant, Tay-Sachs disease type AB, or glycogen storage disease.

34. The method of claim 33, wherein the lysosomal storage disease is Gaucher disease type 1, Gaucher disease type 2, or Gaucher disease type 3.

35. The method of claim 1, wherein the sample comprises blood, plasma, or serum.

36. The method of claim 1, wherein the sample comprises urine, semen, lachrymal fluid, cerebrospinal fluid, vitreous humor, or a tissue sample.

37. The method of claim 1, wherein the denaturant is urea.

38. The method of claim 1, wherein the denaturant is guanadinium hydrochloride, guanadinium sulfite, guanadinium thiocyanate, sodium n-dodecyl sulfate, Nonindet-40 (NP40), or n-lauryl sarcosine.

39. The method of claim 1, wherein the denaturant is high or low pH.

40. The method of claim 1, wherein the denaturant is heat.

41. The method of claim 1, wherein the cross-linking agent is glutaraldehyde.

42. The method of claim 1, wherein the cross-linking agent is succinimidyl acetylthioacetate (SATA), ethylene glycol disuccinate di-(N-succinimidyl) ester (EGS), isocyanate, ultra-violet light, bis-(maleimideo)-methyl ether (BMME), or carbodiimide 1-ethyl-3-(3-dimethyl amino propyl) carbodiimide hydrochloride (EDC).

43. A kit for detecting the presence or amount of stabilized tetrameric transthyretin, comprising:
a denaturant;
a cross-linking agent;
a transthyretin-specific antibody; and
instructions for use in detection of stabilized tetrameric transthyretin.

44. A kit for detecting the presence or amount of stabilized tetrameric transthyretin, comprising:
a denaturant;
a transthyretin-specific antibody that preferentially binds to tetrameric transthyretin as compared to monomeric transthyretin; and
instructions for use in detection of stabilized tetrameric transthyretin.

45. The kit of claim 43, wherein the denaturant is urea.

46. The kit of claim 43, wherein the cross-linking agent is glutaraldehyde.
